(19) United States
(12) Patent Application Publication
(10) Pub. No.: US 2023/0033480 A1
(43) Pub. Date: Feb. 2, 2023

(54) DATA PROCESSING APPARATUS AND INFERENCE METHOD

(71) Applicant: SHIMADZU CORPORATION, Kyoto-shi (JP)

(72) Inventors: Yuriko NAKAKIMURA, Kyoto-shi (JP); Shiori NAGAI, Kyoto-shi (JP); Tomohiro KAWASE, Kyoto-shi (JP); Yusuke YOKOI, Kyoto-shi (JP); Satoshi YAMAMOTO, Kyoto-shi (JP); Kenta ADACHI, Kyoto-shi (JP)

(21) Appl. No.: 17/857,316

(22) Filed: Jul. 5, 2022

(30) Foreign Application Priority Data
Jul. 5, 2021 (JP) 2021-111527
Jun. 23, 2022 (JP) 2022-100940

(51) Int. Cl. G06N 5/04; G06N 20/00

(52) U.S. Cl. CPC G06N 5/04; G06N 20/00

(57) ABSTRACT

A data processing apparatus according to one aspect is provided with an inference unit configured to predict at least one objective variable from a plurality of explanatory variables by using a trained model, and a display data generation unit configured to generate data for displaying an inference result by the inference unit. The inference unit is configured to set a first explanatory variable selected from the plurality of explanatory variables as a variation value and set second explanatory variables other than the first explanatory variable as fixed values. The inference unit predicts, by using the trained model, the at least one objective variable when the first explanatory variable is continuously varied within a predetermined variation range. The display data generation unit generates data indicating a variation of the at least one objective variable with respect to a variation of the first explanatory variable.

### BACKGROUND OF THE INVENTION

### Field of the Invention

The present invention relates to a data processing apparatus and an inference method.

### Description of the Background Art

Japanese Unexamined Patent Application Publication No. 2018-036131 discloses a method of estimating a state of a structural complex from a plurality of parameters acquired by measuring a target structural complex using a trained neural network.

According to the method described in Japanese Unexamined Patent Application Publication No. 2018-036131, when a data set of a plurality of parameters is inputted to the input layer of a neural network, an estimation value of the performance of the structural complex is outputted from the output layer of the neural network.

### SUMMARY OF THE INVENTION

However, in the estimation method described in Japanese Unexamined Patent Application Publication No. 2018-036131, it is merely possible to acquire only one estimation value corresponding to a data set of a plurality of parameters inputted to the neural network. For this reason, it is not considered to be easy for the user to know how each of the plurality of parameters influences the predicted performance from the one estimation value. For example, when values of some parameters among the above-described plurality of parameters are fluctuated (increased or decreased), it is not considered to be easy to predict how the estimation value varies.

To understand how each parameter affects the estimation value, it is necessary to prepare a data set of a plurality of parameters to be inputted to the neural network and repeat the process of acquiring an estimation value for each data set. For this reason, there are concerns that the user's convenience may be lowered and the efficiency of the inference processing may be deteriorated. These concerns may become more pronounced as the number of parameters to be inputted to the neural network increases.

The present invention has been made to solve the above-described problems. It is an object of the present invention to enhance the usefulness of an inference result outputted from a trained model in response to inputs of a plurality of explanatory variables.

A data processing apparatus according to a first aspect of the present invention is provided with an inference unit configured to predict at least one objective variable from a plurality of explanatory variables by using a trained model and a display data generation unit configured to generate data for displaying an inference result by the inference unit. The inference unit is configured to set a first explanatory variable selected from the plurality of explanatory variables as a variation value and set second explanatory variables other than the first explanatory variable as fixed values. The inference unit is configured to predict, by using the trained model, the at least one objective variable when a first explanatory variable is continuously varied within a predetermined variation range. The display data generation unit generates data indicating a variation of the at least one objective variable with respect to a variation of the first explanatory variable.

An inference method according to a second aspect of the present invention predicts at least one objective variable from a plurality of explanatory variables by using a trained model. The inference method includes the steps of: predicting at least one objective variable, by using the trained model, when a first explanatory variable selected from a plurality of explanatory variables is set as a variation value, second explanatory variables other than the first explanatory variable are set as fixed amounts, and the first explanatory variable is continuously varied within a predetermined variation range; generating data indicating a variation of the at least one objective variable with respect to a variation of the first explanatory variable; and displaying the data generated by the step of generating the data.

The above-described objects and other objects, features, aspects, and advantages of the present invention will become apparent from the following detailed descriptions of the present invention that can be understood with reference to the attached drawings.

### BRIEF DESCRIPTION OF THE DRAWINGS

FIG. 1 is a schematic diagram illustrating a configuration example of an analysis system according to Embodiment 1.

FIG. 2 is a diagram schematically illustrating a hardware configuration example of an information processing apparatus and a data processing apparatus.

FIG. 3 is a diagram schematically showing a functional configuration of an information processing apparatus and a data processing apparatus.

FIG. 4 is a flowchart for explaining an outline of the processing performed by the data processing apparatus.

FIG. 5 is a flowchart for explaining the processing procedure of generating (S01 in FIG. 4) a sample list.

FIG. 6 is a diagram showing a configuration example of a sample list.

FIG. 7 is a flowchart for explaining the processing procedure of generating training data (S02 in FIG. 4), machine learning (S03 in FIG. 4), and storing a trained model (S04 in FIG. 4).

FIG. 8 is a diagram showing a configuration example of a training data table.

FIG. 9 is a diagram showing a configuration example of a trained model list.

FIG. 10 is a flowchart for explaining the processing procedure of the inference processing (S05 to S07 in FIG. 4) in the data processing apparatus according to Embodiment 1.

FIG. 11 is a diagram schematically showing a first display example of an inference result in a display unit.

FIG. 12 is a diagram showing a second displaying example of an inference result in the display unit.

FIG. 13 is a diagram schematically showing a third display example of an inference result in the display unit.

FIG. 14 is a flowchart for explaining a processing procedure of inference processing in a data processing apparatus according to Embodiment 2.

FIG. 15 is a flowchart for explaining the processing procedure of generating training data, machine learning, and storing a trained model in the data processing apparatus according to Embodiment 2.

FIG. 16 is a flowchart for explaining the inference processing in the data processing apparatus according to a first configuration example of Embodiment 3.

FIG. 17 is a diagram schematically showing a display example of an inference result in the first configuration example.

FIG. 18 is a flowchart for explaining the inference processing in the data processing apparatus according to a second configuration example of Embodiment 3.

FIG. 19 is a flowchart for explaining the inference processing in the data processing apparatus according to a third configuration example of Embodiment 3.

FIG. 20 is a flowchart for explaining the processing procedure of generating training data, machine learning, and storing a trained model in the data processing apparatus according to Embodiment 4.

FIG. 21 is a diagram showing a configuration example of a sample list.

FIG. 22 is a diagram showing a configuration example of a selection sample extraction table.

FIG. 23 is a diagram showing a configuration example of a trained model list.

FIG. 24 is a flowchart for explaining the processing procedure of the inference processing in the data processing apparatus according to Embodiment 5.

FIG. 25 is a diagram schematically showing a display example of a plurality of inference results in a display unit.

FIG. 26 is a flowchart for explaining the processing procedure for generating a sample list in the data processing apparatus according to Embodiment 5.

### DESCRIPTION OF THE PREFERRED EMBODIMENTS

Hereinafter, some embodiments of the present invention will be described in detail with reference to the attached figures. Note that hereinafter, the same or corresponding portion in the figures will be assigned by the same or corresponding symbol in the figure, and the explanation thereof will not be basically repeated.

### Embodiment 1

[Configuration Example of Analysis System]

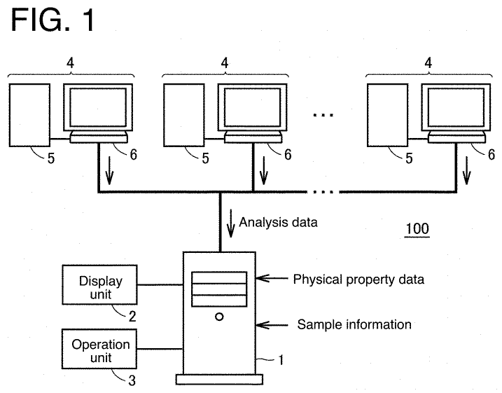

FIG. 1 is a schematic diagram for explaining a configuration example of an analysis system according to Embodiment 1. The analysis system according to Embodiment 1 can be applied to a system for a cross-sectionally analyzing analysis data acquired in a plurality of analyzers.

As shown in FIG. 1, the analysis system 100 according to this embodiment is provided with a plurality of analyzers 4 and a data processing apparatus 1.

The plurality of analyzers 4 performs a measurement of a sample. The plurality of analyzers 4 includes, for example, a liquid chromatograph (LC), a gas chromatograph (GC), a liquid chromatograph mass spectrometer (LC-MS), a gas chromatograph mass spectrometer (GC-MS), a pyrolysis-gas chromatograph mass spectrometer (Py-GC/MS), a scanning electron microscope (SEM), a transmission electron microscope (TEM), an energy dispersive X-ray fluorescence analyzer (EDX), a wavelength dispersive fluorescent X-ray analyzer (WDX), a nuclear magnetic resonator (NMR), and a Fourier transform infrared spectrophotometer (FT-IR). The plurality of analyzers 4 may further include a photodiode array detector (LC-PDA), a liquid chromatograph tandem mass spectrometer (LC/MS/MS), a gas chromatograph tandem mass spectrometer (GC/MS/MS), a liquid chromatograph ion trap time-of-flight mass spectrometer (LC/MS-IT-TOF), a near-infrared spectrometer, a tensile testing machine, a compression testing machine, an emission spectroscopic analyzer (AES), an atomic absorption analyzer (AAS/FL-AAS), a plasma mass spectrometer (ICP-MS), an organic elemental analyzer, a glow discharge mass spectrometer (GDMS), a particle composition analyzer, a trace total nitrogen automatic analyzer (TN), a high-sensitivity nitrogen carbon analyzer (NC), and a thermal analyzer. When the analysis system 100 has a plurality of different types of analyzers 4, it is possible to multilaterally analyze one sample using a plurality of types of analysis data.

The analyzer 4 includes a device body 5 and an information processing apparatus 6. The device body 5 measures a sample serving as an analysis target. To the information processing apparatus 6, the identification information of the sample and the measurement conditions of the sample are inputted.

The information processing apparatus 6 controls the measurement by the device body 5 according to the inputted measurement condition. With this, analysis data based on the measurement result of the sample is acquired. The information processing apparatus 6 stores the acquired analysis data along with the identification information and the measurement condition of the sample in the data file and stores the data file in the built-in memory.

The information processing apparatus 6 is connected to the data processing apparatus 1 so as to be able to communicate with each other. The connectivity between the information processing apparatus 6 and the data processing apparatus 1 can be wired or wireless. For example, the Internet can be used as a communication network between the information processing apparatus 6 and the data processing apparatus 1. With this, the information processing apparatus 6 of each analyzer 4 can transmit the data file for each sample to the data processing apparatus 1.

The data processing apparatus 1 is a device for mainly managing the analysis data acquired by the plurality of analyzers 4. To the data processing apparatus 1, the analysis data from each analyzer 4 is inputted. To the data processing apparatus 1, it is also possible to input the information on the sample (hereinafter also referred to as “sample information”) and the physical property data of the sample.

The sample information includes the identification information (the sample ID, the sample name, etc.) for identifying the sample and the information relating to the production of the sample (hereinafter also referred to as the “recipe data”). The recipe data of the sample may include the information on the blending quantity of the raw materials of the sample and the production process. For example, in a case where the sample is a three-way catalyst, the recipe data includes the blending quantity (g) of Pt (platinum), the blending quantity (g) of Pd (palladium), the stirring time (min), and the firing temperature (° C.), and the like.

The physical property data of the sample denote data indicating the attributes of the sample, which are acquired by an analysis other than the analysis by the analyzer 4. For example, in a case where the sample is a three-way catalyst, the physical property data include, for example, the purification rate (%) of NOx (nitrogen oxide), the purification rate (%) of CO (carbon monoxide), the purification rate (%) of HC (hydrocarbon), and the heat resistance performance.

The data processing apparatus 1 has a built-in database. The database is a storage unit for storing the data exchanged between the data processing apparatus 1 and the plurality of analyzers 4, the data inputted from the outside of the data processing apparatus 1, and the data generated in the data processing apparatus 1. The data processing apparatus 1 stores, for each sample, the data file, the sample information, and the physical property data of the sample in an associated manner in the database. Note that in the example of FIG. 1, the data processing apparatus 1 is configured to include a built-in database, but it may be configured such that a database is externally connected to the data processing apparatus 1.

### [Hardware Configuration Example of Analysis System]

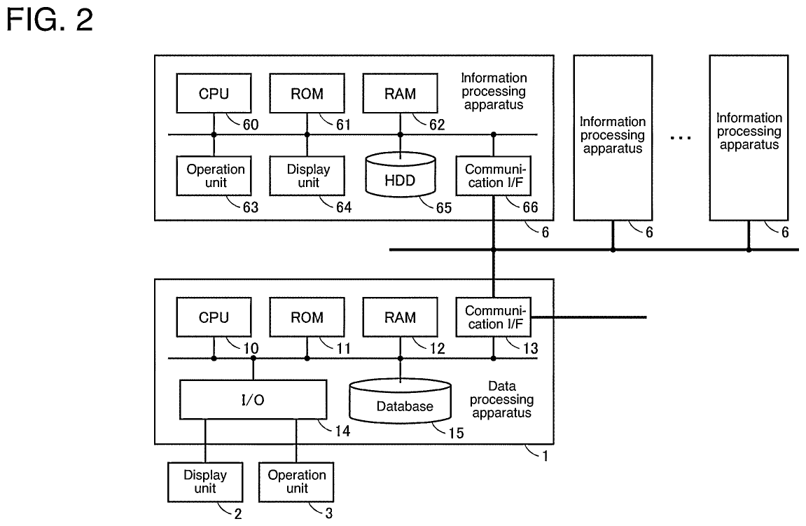

FIG. 2 is a diagram schematically illustrating a hardware configuration example of the information processing apparatus 6 and the data processing apparatus 1.

### (Hardware Configuration of Information Processing Apparatus)

As shown in FIG. 2, the information processing apparatus 6 is provided with a CPU (Central Processing Unit) 60 for controlling the entire analyzer 4 and a storage unit for storing programs and data and is configured to operate in accordance with the programs.

The storage unit includes a ROM (Read Only Memory) 61, a RAM (Random Access Memory) 62, and an HDD (Hard Disk Drive) 65. The ROM 61 stores programs to be executed by the CPU 60. The RAM 62 temporarily stores the data to be used during the execution of the program in the CPU 60. The RAM 62 functions as a temporary data memory used as a working area. The HDD 65 is a non-volatile storage device that stores the information, such as, e.g., a data file, generated per sample by the information processing apparatus 6. In addition to or in place of the HDD 65, a solid-state memory device, such as, e.g., a flash memory, may be adopted.

The information processing apparatus 6 further includes a communication interface (I/F) 66, an operation unit 63, and a display unit 64. The communication I/F 66 is an interface for the information processing apparatus 6 to communicate with external devices including the device body 5 and the data processing apparatus 1.

The operation unit 63 receives an input including an instruction to the information processing apparatus 6 from a user (e.g., an analyst). The operation unit 63 includes a keyboard, a mouse, and a touch panel integrally configured with the display screen of the display unit 64 and receives the measurement condition and the identification information on the sample.

The display unit 64 is capable of displaying, for example, an input screen for a measurement condition and identification information on the sample when setting the measurement condition. During the measurement, the display unit 64 can display the measurement data detected by the device body 5 and the data analysis result by the information processing apparatus 6.

The processing by the analyzer 4 is implemented by each hardware and software executed by the CPU 60. In some cases, such software is pre-stored in the ROM 61 or the HDD 65. Further, software is sometime stored in a storage medium (not shown) and distributed as a program product. Then, the software is read out from the HDD 65 by the CPU 60 and stored in the RAM 62 in a form executable by the CPU 60. The CPU 60 executes this program.

### (Hardware Configuration of Data Processing Apparatus)

The data processing apparatus 1 is provided with a CPU 10 for controlling the entire apparatus and a storage unit for storing programs and data and is configured to operate in accordance with the programs. The storage unit includes a ROM 11, a RAM 12, and a database 15.

The ROM 11 stores programs to be executed by the CPU 10. The RAM 12 temporarily stores the data to be used during the execution of the program in the CPU 10. The RAM 12 functions as a temporary data memory used as a working area.

The database 15 is a non-volatile storage device that stores the data exchanged between the data processing apparatus 1 and the plurality of analyzers 4, the data inputted from the outside of the data processing apparatus 1, and the data generated within the data processing apparatus 1.

The data processing apparatus 1 further includes a communication I/F 13 and an input/output interface (I/O) 14. The communication I/F 13 is an interface that allows the data processing apparatus 1 to communicate with external devices including the information processing apparatus 6.

The I/O 14 is an interface for inputting to the data processing apparatus 1 or for outputting from the data processing apparatus 1. The I/O 14 is connected to a display unit 2 and an operation unit 3. The display unit 2 can show, as will be described later, the information about the processing and a user interface screen for receiving a user's operation when learning processing and inference processing are executed in the data processing apparatus 1.

The operation unit 3 receives inputs including instructions from the user. The operation unit 3 includes a keyboard, a mouse, etc., and receives the sample information, the physical property data of the sample, and the like. Note that the sample information and the physical property data of the sample can be received from an external device via the communication I/F 13.

### [Functional Configuration of Analysis System]

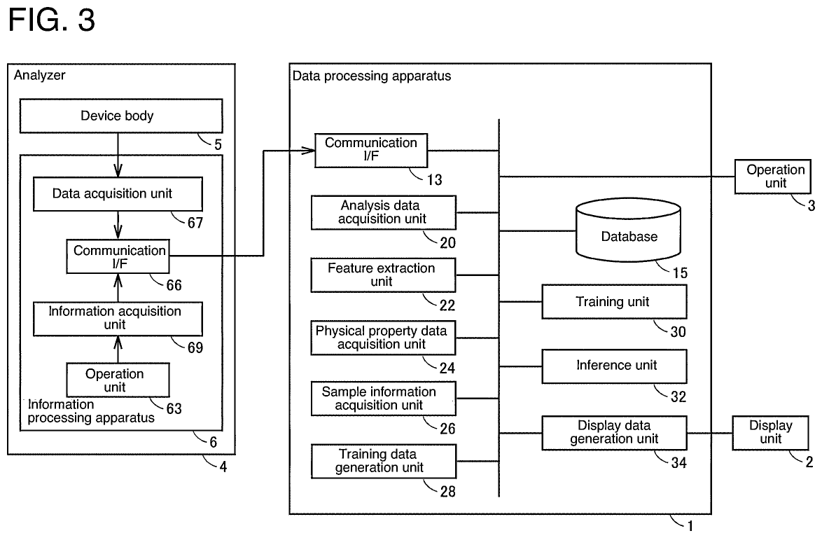

FIG. 3 schematically shows a functional configuration of the information processing apparatus 6 and that of the data processing apparatus 1.

### (Functional Configuration of Information Processing Apparatus)

As shown in FIG. 3, the information processing apparatus 6 includes a data acquisition unit 67 and an information acquisition unit 69. These functional configurations are realized by the CPU 60 executing predetermined programs in the information processing apparatus 6 shown in FIG. 2.

The data acquisition unit 67 acquires the analysis data based on the measurement result of the sample from the device body 5. For example, in a case where the analyzer 4 is a gas chromatograph mass spectrometer (GC-MS), the analysis data includes a chromatogram and a mass spectrum. In a case where the analyzer 4 is a scanning electron microscope (SEM) or a transmission electron microscope (TEM), the analysis data include image data showing a microscopic image of a sample. The data acquisition unit 67 transfers the acquired measurement data to the communication I/F 66.

The information acquisition unit 69 acquires the information received by the operation unit 63. Specifically, the information acquisition unit 69 acquires the sample identification information and the information indicating the measurement condition of the sample. The sample identification information includes, for example, the name of the sample, the name of a product, the model number, the serial number, etc., of a product serving as the sample. The measurement conditions of the sample include device parameters including the name and the model number of the analyzer to be used and measurement parameters indicating the measurement condition, such as, e.g., an application condition of a voltage and/or a current or a temperature condition.

The communication I/F 66 transmits the acquired analysis data, the measurement condition, and the sample identification information to the data processing apparatus 1 as a data file.

### (Functional Configuration of Data Processing Apparatus)

The data processing apparatus 1 is provided with an analysis data acquisition unit 20, a feature extraction unit 22, a physical property data acquisition unit 24, a sample information acquisition unit 26, a training data generation unit 28, a training unit 30, an inference unit 32, and a display data generation unit 34. These functional configurations are realized by the CPU 10 executing predetermined programs in the data processing apparatus 1 shown in FIG. 2.

The analysis data acquisition unit 20 acquires a data file transmitted via the communication I/F 13 from the information processing apparatus 6 of each analyzer 4. The data file includes analysis data of the sample.

The feature extraction unit 22 extracts the feature of the sample by analyzing the analysis data acquired by the analysis data acquisition unit 20 using dedicated data analysis software. The feature of the sample includes, for example, the composition, the concentration, the molecular structure, the number of molecular, the molecular formula, the molecular weight, the degree of polymerization, the particle diameter, the particle area, the number of particles, the dispersity of particles, the peak intensity, the peak area, the slope of the peak, the compound concentration, the compound amount, the absorbance, the reflectance, the transmittance, the sample test strength, the Young's modulus, the tensile strength, the deformation amount, the strain amount, the breaking time, the average interparticle distance, the dielectric tangent, the elongation, the spring hardness, the loss coefficient, the glass transition temperature, and the thermal expansion coefficient of the sample.

The physical property data acquisition unit 24 acquires the physical property data of the sample received by the operation unit 3. The physical property data of the sample is data indicating the attributes of the sample and includes, for example, the value indicating the performance of the sample or the value (e.g., the number of years used) indicating the degree of deterioration of the sample.

The sample information acquisition unit 26 acquires the sample information received by the operation unit 3. The sample information includes the identification information of the sample (the sample ID, the sample name, etc.) and the recipe data of the sample. The recipe data of the sample includes the information on the blending quantity of the raw materials and the production process of the sample.

In the database 15, the analysis data acquired by the analysis data acquisition unit 20, the feature extracted by the feature extraction unit 22, the physical property data acquired by the physical property data acquisition unit 24, and the sample information acquired by the sample information acquisition unit 26 are stored in association with each other for each sample. Specifically, the sample list is generated in the database 15 based on the information. Note that the sample list is a group of data sets generated in accordance with the type of the project or the sample, and its configuration or the like is not particularly limited.

The training data generation unit 28 generates training data (learning data) based on the data stored in the database 15 in response to the user's input operation to the operation unit 3. Note that training data is data in which inputs (explanatory variables) and outputs (objective variables) are set.

For example, the training data generation unit 28 can generate training data in which, for example, the “analysis data or the feature” and/or “the recipe data” of one sample is set as inputs (explanatory variables) of a prediction model, and the “physical property data” of the sample is set as an output (objective variable) of the prediction model.

Alternatively, the training data generation unit 28 can generate training data in which the “recipe data” of one sample is set as inputs (explanatory variables) of a prediction model, and the “analysis data or the feature” or the “physical property data” of the sample is set as an output (objective variable) of the prediction model.

Alternatively, the training data generation unit 28 can generate training data in which the “physical property data” of one sample is set as inputs (explanatory variables) of a prediction model, and the “analysis data or the feature” or the “recipe data” of the sample is set as an output (objective variable) of the prediction model.

The generated training data is provided to the training unit 30. The training data may be stored in the database 15 each time it is generated. As a result, the training data is accumulated in the database 15.

Note that the training data generation unit 28 displays the confirmation screen for confirming whether or not the training data is to be stored in the database 15 using the display data generation unit 34 on the display unit 2, before storing the training data in the database 15. In a case where the confirmation screen accepts a user operation for instructing to store the training data, the training data generation unit 28 stores the training data in the database 15. On the other hand, in a case where the confirmation screen does not accept the user operation, the training data generation unit 28 discards the training data.

The training unit 30 performs supervised learning in which the explanatory variables of the training data are set as inputs of a prediction model and the objective variable of the training data is set as an input of the ground truth data of the output of the prediction model, by using the training data generated by the training data generation unit 28. In the supervised learning, it predicts that a given input produces what outputs. The method of the machine learning using the training data in the training unit 30 is not particularly limited, and for example, a known machine learning, such as, e.g., a neural network (Neural Network; NN) and a support vector machine (SVM), can be used.

When the learning is completed, a trained model is acquired. The generated trained model is stored in the database 15. Specifically, the trained model is stored in the database 15 in association with the identification information for identifying the trained model, the generation date and time of the trained model, and the identification information for identifying the training data used for the learning.

The inference unit 32 predicts an output (objective variable) from the input data (explanatory variables) newly inputted from any one or two of the analysis data acquisition unit 20, the feature extract unit 22, the physical property data acquisition unit 24, and the sample information acquisition unit 26, using the trained model stored in the database 15. That is, the explanatory variable is one or two of the “analysis data or the feature,” the “physical property data,” and the “recipe data,” and the objective variable is the other one of the “analysis data or the feature,” the “physical property data,” and the “recipe data.” Alternatively, the explanatory variable is one of the “analysis data or the feature,” the “physical property data,” and the “recipe data,” and the objective variable is the other one or two of the “analysis data or the feature,” the “physical property data,” and the “recipe data.” In one example, the explanatory variable is the “analysis data or the feature” and/or the “recipe data,” and the objective variable is the “physical property data.”

Upon acquiring the inference result by the inference unit 32, the display data generation unit 34 generates data for displaying the inference result on the display screen of the display unit 2. Further, the display data generation unit 34 displays the information about the processing when the learning processing and the inference processing are executed and provides a user interface (UI) for receiving an operation of the user.

Note that it may be configured such that, in place of the operation unit 3 and the display unit 2, for example, an information terminal, such as, e.g., a desktop personal computer (PC), a notebook PC, and a portable terminal (a tablet terminal, a smartphone), is connected to the data processing apparatus 1.

Further, in the example shown in FIG. 3, although the data processing apparatus 1 is provided with the feature extraction unit 22, it may be configured such that the information processing apparatus 6 is provided with the feature extraction unit 22. In this case, the information processing apparatus 6 transmits the feature to the data processing apparatus 1 together with the analysis data of the sample.

### [Operation of Data Processing Apparatus]

Next, the processing performed by the data processing apparatus 1 will be described.

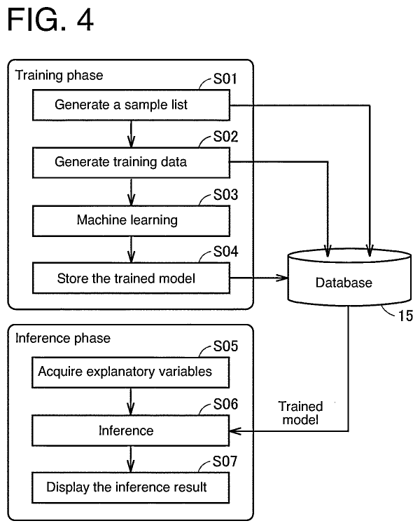

FIG. 4 is a flowchart for explaining the outline of the processing performed by the data processing apparatus 1. As shown in FIG. 4, the processing in the data processing apparatus 1 can be roughly classified into a training phase and an inference phase.

### <Training Phase>

In the training phase, training data is generated using the data stored in the database 15. Then, supervised learning is executed using the generated training data to generate a trained model.

As shown in FIG. 4, first, in Step (hereinafter simply referred to as “S”) 01, a sample list is generated based on the data stored in the database 15. The sample list denotes a group of data sets generated according to the type of the project or the sample.

Then, in S02, training data is generated from the sample list in response to the input operation by the user to the operation unit 3. The generated training data is stored in the database 15.

Next, in S03, using the training data, supervised learning is performed in which explanatory variables of the training data are used as inputs of a prediction model and objective variables of the training data are used as ground truth data of outputs of the prediction model. Finally, in S04, the trained model generated by the supervised learning is stored in the database 15.

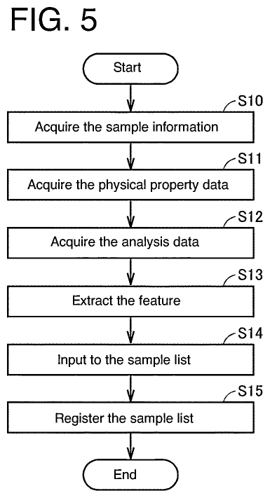

Hereinafter, with reference to FIGS. 5 to 9, specific processing in the training phase will be described.

### (1) Generation of Sample List (S01 In FIG. 4)

FIG. 5 is a flowchart for explaining the processing procedure of generating (S01 of FIG. 4) a sample list. Referring to FIG. 5, in S10, sample information is acquired via the operation unit 3. Specifically, the user can input sample information by using database operating software (frontend) (not shown). The sample information includes the identification information (sample ID, sample name, etc.) of the sample and the recipe data of the sample. The recipe data of the sample includes the information on the blending quantity of the raw materials of the sample and the production process of the sample.

In S11, the physical property data of the sample is acquired via the operation unit 3. The physical property data is data indicating the attributes of the sample.

In S12, a data file transmitted from the information processing apparatus 6 of each analyzer 4 is acquired via the communication I/F 13. The data file includes the analysis data of the sample.

In S13, the feature of the sample is extracted by analyzing the analysis data acquired in S12 using dedicated data analysis software.

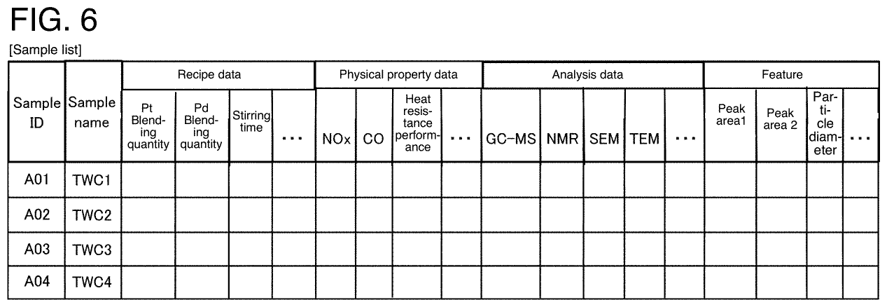

In S14, the acquired sample information (the identification information of the sample and the recipe data), the physical property data of the sample, and the analysis data and the feature of the sample are inputted to the sample list. FIG. 6 shows a configuration example of a sample list. FIG. 6 shows a configuration example of a sample list in a case where the sample is a three-way catalyst.

As shown in FIG. 6, in the sample list, the sample name, the recipe data, the physical property data, the analysis data, and the feature of the sample are inputted in association with the sample ID. The recipe data includes the blending quantity (g) of Pt, the blending quantity (g) of Pd, the stirring time (min), the firing temperature (° C.), and the like. The physical property data includes the purification rate (%) of NOx, the purification rate (%) of CO, the purification rate (%) of HC, the heat resistance performance, and the like. The analysis data includes analysis data acquired by a gas chromatograph mass spectrometer (GC-MS), a nuclear magnetic resonator (NMR), a scanning electron microscope (SEM), a transmission electron microscope (TEM), and the like.

The feature includes a peak area with respect to a predetermined mass number acquired by analyzing a chromatogram acquired by a gas chromatograph mass spectrometer (GC-MS), an abundance ratio of a predetermined substance acquired by analyzing an NMR spectrum acquired by a nuclear magnetic resonator (NMR), a particle diameter of a particle present in a ternary catalyst, acquired by analyzing an SEM image acquired by a scanning electron microscope (SEM), an average particle diameter, and a particle diameter of a particle present in a ternary catalyst, acquired by analyzing a TEM image acquired by a transmission electron microscope (TEM), and the like.

The sample list in which the recipe data, the physical property data, the analysis data, and the feature of the sample are inputted is given by information (such as the name of the sample list) for identifying the sample list and registered in the database 15.

### (2) Generation of Training Data and Generation of Trained Model (S02 to S04 in FIG. 4)

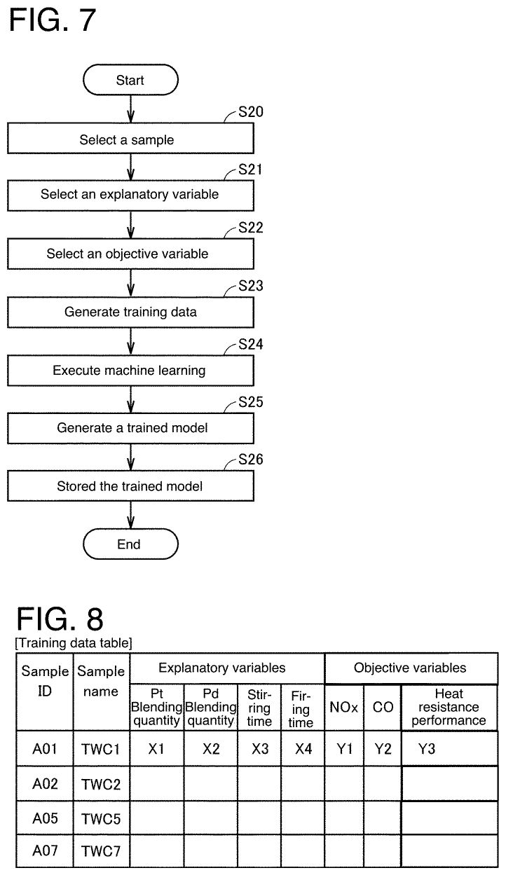

FIG. 7 is a flowchart for explaining the processing procedure of generating the training data (S02 in FIG. 4), the machine learning (S03 in FIG. 4), and storing the trained model storage (S04 in FIG. 4).

Referring to FIG. 7, first, in S20, a sample to be used for generating training data is selected. The user can select a sample by operating a UI screen (sample selection screen) displayed on the display unit 2 using the operation unit 3.

This sample selection screen can be generated based on the sample list stored in the database 15. For example, in the sample selection screen, a list including the sample name, the recipe data, etc., is displayed for all samples stored in the database 15. In this case, selection icons corresponding to the respective samples are displayed on the sample selection screen. The user can select any sample by checking the selection icon using the operation unit 3.

Alternatively, names of a plurality of sample lists stored in the database 15 may be displayed on the sample selection screen. In this case, selection icons corresponding to the respective sample lists are displayed on the sample selection screen. When the user checks the selection icon using the operation unit 3, all samples included in the corresponding sample list are selected.

When the sample selection is completed, the data included in the row of the selected sample is extracted from the sample list, and a selection sample extraction table is generated. It may be configured such that the selection sample extraction table is displayed on the display unit 2 in order to confirm the selection result.

Then, in S21 and S22, the explanatory variables and the objective variables to be used to generate training data are selected. On the display unit 2, UI screens (explanatory variable selection screen and objective variable selection screen) are displayed.

The explanatory variable selection screen is a UI screen for the user to select the type of data used to input training data. On the explanatory variable selection screen, the types of the recipe data, the analysis data, and the feature of the sample included in the selection sample extraction table are listed and displayed. For example, in a case where the selected sample is a three-way catalyst, on the explanatory variable selection screen, the type of the recipe data, such as, e.g., the “blending quantity of platinum (Pt)” and the “blending quantity of palladium (Pd),” the type of the analysis data, such as, e.g., “GC-MS” and “NMR,” and the type of the feature, such as, e.g., the “peak area” and the “particle diameter,” are displayed. On the explanatory variable selection screen, selection icons are displayed corresponding to the respective types. The user can select any explanatory variable by checking a selection icon using the operation unit 3.

The objective variable selection screen is a UI screen for the user to select the type of data to be used for outputting training data. On the objective variable selection screen, the list of the type of the recipe data, the analysis data, and the feature of the sample included in the selection sample extraction table is displayed. Selection icons are displayed on the objective variable selection screen for each type. The user can select any objective variable by checking the selection icon with the operation unit 3.

However, in the objective variable selection screen, for the type belonging to the same category as the type of data selected for the explanatory variable, no selection icon is displayed to avoid a duplicate selection. Therefore, for example, in a case where a type belonging to the “recipe data” is selected as an explanatory variable, the type belonging to either the “physical property data” or the “analysis data or the feature” can be selected as an objective variable. Alternatively, in a case where a type belonging to the “physical property data” is selected as an explanatory variable, the type belonging to either the “recipe data” or the “analysis data or the feature” can be selected as an objective variable. Alternatively, in a case where a type belonging to the “analysis data or the feature” is selected as an explanatory variable, the type belonging to either the “recipe data” or the “physical property data” can be selected as an objective variable.

Note that the training data generating application may be prepared for each set of data to be used as an explanatory variable and data to be used as an objective variable of supervised learning. In this case, the user can select the type of data to be used for inputting and outputting training data only by performing the selection of the training data generation application.

When the selection of the explanatory variable (the type of input data) and the objective variable (the type of output data) is completed, in S23, a training data table is generated by extracting the data that matches the explanatory variable and the data that matches the objective variable from the selection sample extraction table. FIG. 8 is a diagram showing a configuration example of a training data table. FIG. 8 shows a configuration example of a training data table in a case where the selected sample is a three-way catalyst.

As shown in FIG. 8, the sample IDs and the sample names selected in S20 of FIG. 7 are displayed vertically. Further, the explanatory variables (the types of input data) selected in S21 and the objective variables (the types of output data) selected in S22 of FIG. 7 are displayed horizontally.

In the example of FIG. 8, recipe data are selected as explanatory variables. Specifically, the blending quantity (%) of Pt, the blending quantity (%) of Pd, the stirring time (min), and the firing temperature (° C.) are selected as explanatory variables. Physical property data are selected as objective variables. Specifically, the purification rate (%) of NOx, the purification rate (%) of CO, and the heat resistance performance are selected as objective variables.

In the training data table, data matching the explanatory variables and data matching the objective variables are inputted for each sample. The training data table is displayed on the display unit 2. The user can add or modify the sample and the type of data to the displayed training data table. For example, in a case where a sample is added, the sample is added to the selection sample extraction table, and the data of the added sample is added in the training data table. In a case where the type of data of the explanatory variable or the objective variable is added, in the training data table, the data of the added variable is added to each sample.

When the generation of the training data table is completed, training data is generated based on the generated training data table. In the example of FIG. 8, training data is generated in which the blending quantity (g) of Pt, the blending quantity (g) of Pd, the stirring time (min), and the firing temperature (° C.) selected as explanatory variables are inputs, and the purification ratio (%) of NOx, the purification ratio (%) of CO, and the heat resistance performance selected as objective variables are outputs.

Next, in S24, supervised learning is performed by using the training data in which the explanatory variables of the training data are inputs of the learning model and the objective variables of the training data are ground truth data of outputs of the model. Hereinafter, a support vector machine (SVM) will be described as an example of machine learning.

The training unit 30 (FIG. 3) inputs the blending quantity (g) of Pt, the blending quantity (g) of Pd, the stirring time (min), and the firing temperature (° C.) to the SVM and acquires the purification rate (%) of NOx, the purification rate (%) of CO, and the heat resistance performance outputted from the SVM. The training unit 30 compares the purification rate (%) of NOx, the purification rate (%) of CO, and the heat resistance performance acquired as described above with the purification rate (%) of NOx, the purification rate (%) of CO, and the heat resistance performance included in the training data, respectively. The training unit 30 generates a trained model by updating various parameters in the SVM such that the purification rate (%) of NOx, the purification rate (%) of CO, and the heat resistance performance outputted from the SVM approach the purification rate (%) of NOx, the purification rate (%) of CO, and the heat resistance performance of the training data, respectively.

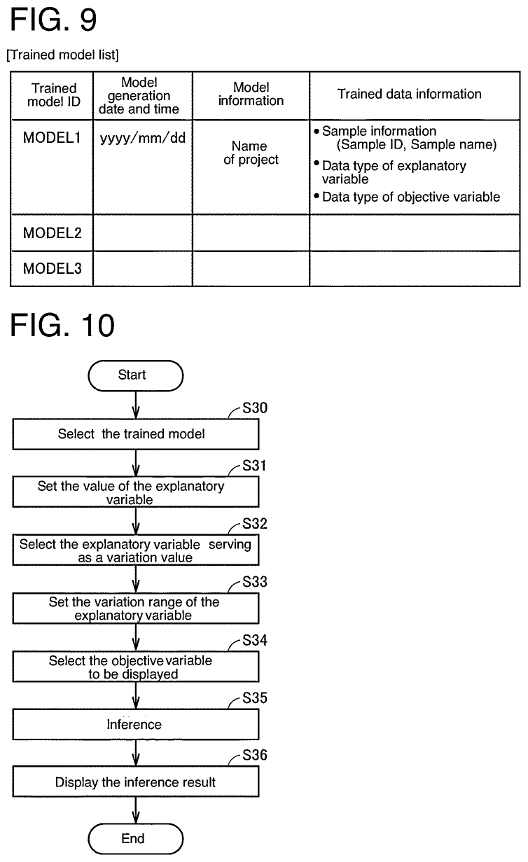

When the machine learning in S24 is completed, a trained model is acquired (S25 of FIG. 7). In S26, the generated trained model is stored in the database 15. Specifically, the trained model is registered in the trained model list stored in the database 15. FIG. 9 shows a configuration example of a trained model list. In the example of FIG. 9, the trained model is associated with the identification information (e.g., trained model ID) to identify the trained model, the generation date and time of the trained model, the information about the trained model, and the identification information to identify the training data used for the learning.

The information on the trained model may include the name, etc., of the project to which the trained model is applied. For example, information, such as, e.g., “model for improving the purification performance of the three-way catalyst,” “model for improving the heat resistance of the three-way catalyst,” and the like, may be included. In the identification information on the training data, the sample information (e.g., the sample ID and the sample name) used to generate the training data, the type of data selected for the explanatory variable, the type of data selected for the objective variable, etc., may be included. The information may be acquired from the selection sample data extraction table and the training data table.

### <Inference Phase>

In the inference phase, objective variables are predicted from the given explanatory variables using the generated trained model. The inference result is displayed on the display unit 2. Returning to FIG. 4, first, in S05, explanatory variables are newly acquired. The explanatory variable is one or two of the “analysis data or the feature,” the “physical property data,” and the “recipe data.”

In S06, the trained model receives inputs of the explanatory variables acquired in S05 to predict objective variables. The objective variable to be predicted is the other one or two of the “analysis data or the feature,” the “physical property data,” and the “recipe data.”

In S07, the inference result by the trained model is displayed on the display unit 2. This allows the user to confirm the value of the objective variable predicted from the explanatory variables.

However, in the above-described configuration, only one inference result is acquired for the newly acquired explanatory variables, and therefore, it is considered that it is not easy for the user to know how the explanatory variable affects the predicted object variable from the one inference result. For example, it is considered that it is not easy to predict how the value of the objective variable varies in a case where the value of one explanatory variable is increased (or decreased), from the acquired one inference result.

Therefore, in this embodiment, the inference processing capable of enhancing the usefulness of the inference result will be described. Hereinafter, the specific inference processing will be described with reference to FIGS. 10 to 12.

FIG. 10 is a flowchart for explaining the processing procedures of the inference processing (S05 to S07 in FIG. 4) in the data processing apparatus according to this embodiment.

Referring to FIG. 10, in S30, a trained model to be used for the inference processing is selected. The UI screen (trained model selection screen) is displayed on the display unit 2. The trained model selection screen is generated based on the trained model list stored in the database 15 (FIG. 9). In the trained model selection screen, together with the identification information (the trained model ID, etc.) of the trained model included in the trained model list (FIG. 9), the generation date and time of the trained model, the information (the name of the project, etc.) on the trained model, and the identification information for identifying the training data used for the learning are displayed.

In the trained model selection screen, selection icons are displayed corresponding to the respective trained models. The user can select any trained model by checking the selection icon with the operation unit 3.

By selecting the trained model to be used for the inference processing, the type of data of explanatory variables to be inputted to the trained model and the type of data of objective variables to be predicted by the trained model are determined. The data of the explanatory variable is one of the “recipe data,” the “physical property data,” and the “analysis data or the feature.” The data of the objective variable is one or two of the other of the “recipe data,” the “physical property data,” and the “analysis data or the feature.” Alternatively, the data of explanatory variable is two of the “recipe data,” the “physical property data,” and the “analysis data or the feature.” The data of the objective variable is the other of the “recipe data,” the “physical property data,” and the “analysis data or the feature.”

Specifically, in the trained model list (FIG. 9), the identification information on the training data used to generate the model is registered in association with the model for each trained model. As mentioned above, the identification information on the training data includes the sample information (the sample ID, the sample name, etc.) used for generating the training data, the type of data selected for the explanatory variable, and the type of data selected for the object variables. Thus, by selecting a trained model, the type of data of the explanatory variable to be inputted to the trained model and the type of data of the objective variable to be predicted by the trained model can be automatically determined.

Next, in S31, the value of the explanatory variable to be inputted to the trained model is set. On the display unit 2, a UI screen (sample selection screen) for selecting a sample to be analyzed is displayed. The user can select a sample to be analyzed by operating the sample selection screen with the operation unit 3.

This sample selection screen can be generated based on the sample list (FIG. 6) stored in the database 15. For example, in the sample selection screen, a list including the sample name and the recipe data is displayed for all samples stored in the database 15. In the sample selection screen, a selection icon is further displayed corresponding to each sample. The user can select any sample by checking the selection icon using the operation unit 3.

When a sample to be analyzed is selected, a value of an explanatory variable to be inputted to the trained model is set, based on the recipe data, the physical property data, the analysis data, and the feature of the selected sample. Note that the user can adjust each explanatory variable to a desired value by performing an operation for increasing or decreasing the set value as a reference value from the reference value using the operation unit 3.

Next, in S32, the “explanatory variable serving as a variation value” is selected from a plurality of explanatory variables. In the inference processing according to this embodiment, some of a plurality of explanatory variables to be inputted to the trained model are variation values, and the remaining explanatory variables are fixed values. Note that the number of some partial explanatory variables may be 1 or 2 or more. As will be described later, the user can select an explanatory variable serving as a variation value by operating the user interface screen displayed on the display unit 2 using the operation unit 3.

The “explanatory variable serving as a variation value” denotes an explanatory variable whose value varies within a predetermined variation range in the inference processing. On the other hand, the “explanatory variable serving as a fixed value” denotes an explanatory variable whose value is fixed in the inference processing.

In S33, the variation range is set for the explanatory variable serving as a variation value. As will be described later, the user can set a variation range of the explanatory variable by inputting the upper limit value and the lower limit value of the variation range by using the operation unit 3 to the user interface screen displayed on the display unit 2. Note that the variation range of the explanatory variable may be automatically set by the data processing apparatus 1 based on the sample list (FIG. 6) stored in the database 15. For example, it may be configured such that data corresponding to the explanatory variable serving as a variation value is extracted from the training data used to generate the trained model, the minimum value among the extracted data is set to the lower limit value in the recommendation range and that the maximum is set to the upper limit value in the recommendation range.

In S34, an objective variable to be displayed is selected from the objective variables predicted from the trained model. The user can select an objective variable to be displayed by operating the user interface screen (objective variable selection screen) displayed on the display unit 2 using the operation unit 3. The number of the objective variables to be displayed may be 1 or 2 or more.

In this objective variable selection screen, the type of data of the objective variable to be predicted by the trained model selected in S30 is displayed. In the objective variable selection screen, selection icons are further displayed corresponding to the respective objective variables. The user can select any objective variable to be displayed by checking the selection icon with the operation unit 3.

Next, in S35, an objective variable is predicted by inputting the explanatory variables set in S31 to S33 to the trained model selected in S30. In this inference processing, an objective variable is predicted corresponding to each value of some continuously varying explanatory variables among the plurality of explanatory variables. In other words, it is predicted how the objective variable varies due to the variation of some explanatory variables.

In S36, the inference result acquired by the inference processing in S35 is displayed on the display unit 2. On the display unit 2, a graph showing the variation with respect to the variation of the above-mentioned some explanatory variables is displayed for the objective variable selected as the object target in S34.

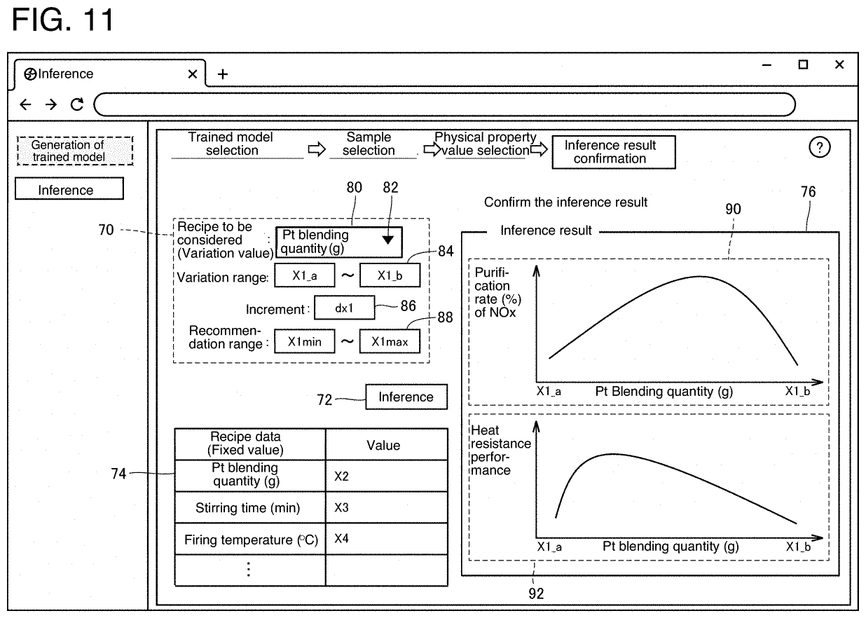

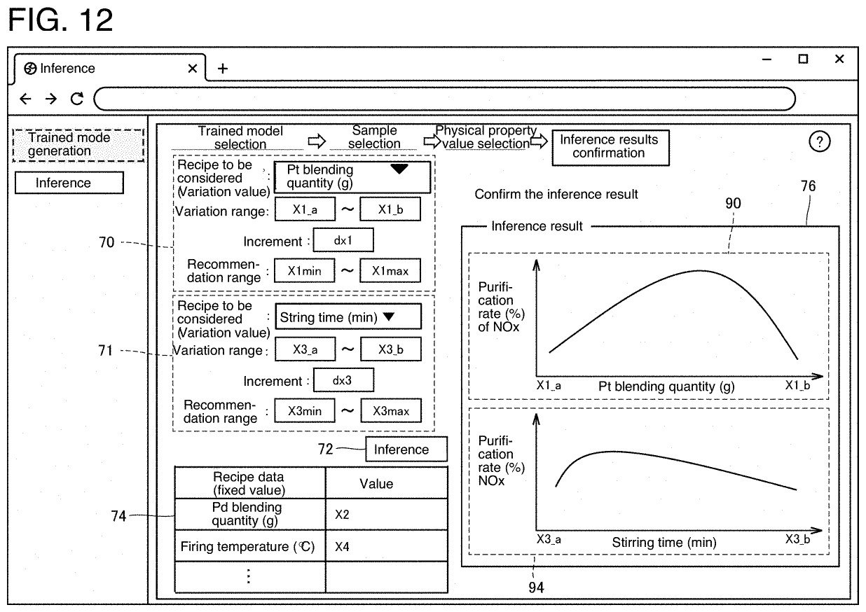

Next, display examples of the inference results in the display unit 2 will be described with reference to FIGS. 11 and 12.

FIG. 11 is a diagram schematically showing a first display example of the inference results in the display unit 2. In FIG. 11, it exemplifies inference results to be displayed on the display unit 2 in a case where the “recipe data” is selected as the explanatory variables to be inputted to the trained model and the “physical property data” are selected as the objective variables to be predicted. The sample is a three-way catalyst.

The display example of FIG. 11 is generated by performing a selection of a trained model used for the inference processing, a selection of a sample to be analyzed, and a selection of an objective variable to be displayed.

As shown in FIG. 11, in the display unit 2, a GUI (Graphical User Interface) 70 is displayed. The GUI 70 is a GUI for selecting an explanatory variable serving as a variation value among a plurality of explanatory variables to be inputted to the trained model. Specifically, the GUI 70 includes a GUI 80 for selecting an explanatory variable serving as a variation value, a GUI 84 for setting a variation range of the explanatory variable, and a GUI 86 for setting the increment of the explanatory variable in the variation range. By operating the GUIs 80, 84, and 86 using the operation unit 3, the user can select the explanatory variable serving as a variation value and set the variation range and the increment of the explanatory variable.

In the right-hand corner of the GUI 80, there is an icon 82 for selecting an explanatory variable serving as a variation value. When the user clicks the icon 82 using the operation unit 3, a GUI (not shown) for displaying explanatory variable candidates serving as variation values is displayed on the lower side of the GUI 80. In this GUI, a list of types of data of a plurality of explanatory variables associated with the selected trained model is displayed. When the user selects an explanatory variable serving as a variation value among a plurality of explanatory variables in the GUI, in the GUI 80, the type of data of the selected explanatory variable is written. In the case of FIG. 11, the “blending quantity (g) of Pt” is selected as the explanatory variable serving as a variation value.

The GUI 84 is configured to be capable of inputting the lower limit value and the upper limit value of the variation range for the explanatory variable serving as a variation value. The GUI 86 is configured to be capable of inputting an increment when the explanatory variable serving as a variation value is continuously varied. In the GUI 84, the user can set the variation range of the explanatory variable serving as a variation value. In the GUI 86, the user can set the increment of the explanatory variable serving as a variable value. In the example of FIG. 11, the lower limit value X1_a and the upper limit value X1 b of the variation range of the “blending quantity (g) of Pt” and the increment dxl have been set.

Note that the GUI 70 may include a GUI 88 for indicating the recommendation range of the variation range for the explanatory variable serving as a variation value. This recommendation range can be set based on the training data used to generate the trained model. For example, it is possible to extract the data (for example, the blending quantity of Pt (g)) corresponding to the explanatory variable serving as a variation value from the training data, set the minimum value X1 min of the extracted data to the lower limit value of the recommendation range, and set the maximum value X1 max to the upper limit value of the recommendation range. This allows the user to set a variation range in the GUI 84 while referring to the recommendation range shown in the GUI 88.

In the display unit 2, a GUI 74 for setting values of explanatory variables serving as fixed values among a plurality of explanatory variables to be inputted to the trained model is displayed. The GUI 74 displays the types of data of the explanatory variables serving as fixed values and the value of each data in a table format. In the example of FIG. 11, in the GUI 74, the values X2, X3, and X4 of data of the explanatory variables (the “blending quantity (g) of Pd,” the “stirring time (min),” the “firing temperature (° C.))” other than the “the blending quantity (g) of Pt” among the plurality of explanatory variables to be inputted to the trained model. The values X2, X3, and X4 are set based on the data of the sample to be analyzed.

After each of the values of the plurality of explanatory variables has been set in the above-described procedures, when a GUI 72 for instructing to execute inference processing is clicked, the inference processing is executed. In the inference processing, the explanatory variable serving as a variation value is continuously varied at predetermined increments, and the objective variable corresponding to each value of the explanatory variable is predicted by using a trained model.

The inference results are displayed in the display area 76 of the display unit 2. As shown in FIG. 11, in the display area 76, graphs 90 and 92 showing the relation between the explanatory variable serving as a variation value and the objective variable are displayed. Note that in the example of FIG. 11, the “purification rate (%) of NOx” and the “heat resistance performance” are selected as objective variables (physical property data) to be displayed.

The graph 90 is a two-dimensional graph in which the explanatory variable (blending quantity (g) of Pt) serving as a variation value is represented by the horizontal axis, and the objective variable to be displayed (purification rate (%) of NOx) is represented by the vertical axis. The graph 92 is a two-dimensional graph in which the explanatory variable (blending quantity (g) of Pt) serving as a variation value is represented by the horizontal axis, and the objective variable (heat resistance performance) to be displayed is represented by the vertical axis.

Each of the graphs 90 and 92 shows how the objective variable varies when the explanatory variable serving as a variation value is continuously varied within a predetermined variation range at predetermined increments. According to the graph 90, it can be seen that the purification rate of NOx increases as the blending quantity of Pt increases, while the purification rate of the NOx decreases as the blending quantity of Pt increases when the blending quantity of Pt exceeds a certain value. According to the graph 92, it can be seen that the heat resistance performance increases as the blending quantity of Pt increases, but the heat resistance performance decreases when the blending quantity of Pt exceeds a certain value. Further, comparing the graphs 90 and 92, it can be seen that the blending quantity of Pt when the purification rate of NOx reaches a peak and the blending quantity of Pt when the heat resistance performance reaches a peak are different.

As will be understood from the above, by referring to the inference results displayed on the display unit 2, the user can easily predict how the value of the objective variable varies when one explanatory variable is continuously varied. For example, according to the graphs 90 and 92, it is possible to predict the blending quantity of Pt suitable for realizing a three-way catalyst having a desired physical property.

Note that the user can acquire graphs 90 and 92 corresponding to various explanatory variables by executing inference processing again by changing the value of the explanatory variable serving as a fixed value in the GUI 74. Further, for the other explanatory variables, the user can acquire a graph showing the variation of the objective variable with respect to the continuous variation of the explanatory variable by executing the inference processing again by changing the type, the variation range, and the increment of the explanatory variable serving as the variation value in the GUI 70.

The data indicating the inference results (the raw data acquired from the inference processing and the data of the graphs 90 and 92) are stored in the database 15, along with the information for identifying the trained model used for the inference processing and the information on the explanation variables inputted to the trained model. Further, the data indicating the inference results to be stored in the database 15 can be outputted from the data processing apparatus 1 to an external device via the communication I/F 13. The output format may be, for example, a CSV (Comma-Separated Values) format, a format displayable by related software, such as, e.g., other AI software, statistical analysis software, or the like.

FIG. 12 is a diagram showing a second display example of inference results on the display unit 2. Similar to FIG. 11, FIG. 12 illustrates inference results displayed on the display unit 2 in a case where the “recipe data” is selected as an explanatory variable to be inputted to the trained model and the “physical property data” is selected as an objective variable to be predicted. The sample is a three-way catalyst. The display example of FIG. 12 is generated by performing the selection of the trained model used for the inference processing, the selection of the sample to be analyzed, and the selection of the objective variable to be displayed.

In the display example of FIG. 12, the number of the explanatory variables as serving as variation values is two. On the display unit 2, GUIs 70 and 71 for selecting the explanatory variables serving as variation values are displayed. The configuration of the GUI 70, 71 is the same as that of the GUI 70 shown in FIG. 11. Therefore, in each of the GUI 70 and the GUI 71, the user can select the explanatory variable serving as a variation value and set the variation range and the increment for the selected explanatory variable.

In the example of FIG. 12, as the explanatory variable serving as a variation value, the “blending quantity (g) of Pt” and the “stirring time (min) of Pt” are selected. The lower limit value X1_a and the upper limit value X1 b of the variation range and the increment dxl are set for the “blending quantity (g)” of Pt, and the lower limit value X3_a and the upper limit value X3 b of the variation range and the increment dx3 are set for the “stirring time (min).”

The GUI 74 shows the values X2 and X4 of the data (blending quantity (g) of Pd, the “firing temperature (° C.),” and the like) of explanatory variables other than the “blending quantity (g) of Pt” and the “stirring time (min)” among a plurality of explanatory variables to be inputted to the trained model.

After each value of the plurality of explanatory variables has been set in the above-described steps, when a GUI 72 for instructing the execution of inference processing is clicked, the inference processing is executed. In the inference processing, one of the explanatory variables between the two explanatory variables serving as variable values is continuously varied, and the other explanatory variable is set as a fixed value (e.g., the median of the variation range). By using the trained model, the objective variable corresponding to each value of one of the explanatory variables is predicted. In the example of FIG. 12, the inference processing when the “blending quantity (g) of Pt” among the plurality of explanatory variables is set as a variable value and the remaining explanatory variables are set as fixed values, and the inference processing when the stirring time (min) among the plurality of explanatory variables is set as a variable value and the remaining explanatory variables are set as fixed values are executed.

The inference results are displayed in the display area 76 of the display unit 2. In the display area 76, the graphs 90 and 94 showing the relation between the explanatory variable serving as a variation value and the objective variable to be displayed are displayed. In the example of FIG. 12, as an objective variable (physical property data) to be displayed, the “purification rate (%) of NOx” is selected.

The graph 90 is a two-dimensional graph in which the explanatory variable (the blending quantity (g) of Pt) serving as a variation value is represented by the horizontal axis, and the objective variable to be displayed (the purification rate (%) of NOx) is represented by the vertical axis. The graph 94 is a two-dimensional graph in which the explanatory variable (stirring time (min)) serving as a variation value is represented by the horizontal axis, and the objective (the purification rate (%) of NOx) to be displayed is represented by the vertical axis.

According to the graph 90, it can be seen that the purification rate of NOx increases serving as the blending quantity of Pt increases, while the purification rate of NOx decreases as the blending quantity of Pt increases when the blending quantity of Pt exceeds a certain value. According to the graph 94, the purification rate of NOx increases as the stirring time increases, but when the stirring time exceeds a certain value, the purification rate of NOx decreases. Further, by comparing the graphs 90 and 94, it is possible to know the degree of effect on one objective variable for a plurality of explanatory variables to be inputted to the trained model.

As described above, according to the data processing apparatus of Embodiment 1, the user can easily know how the objective variable varies based on the displayed data when the first explanatory variable serving as a variation value is continuously varied. Therefore, the effectiveness of the inference results can be improved.

Further, in Embodiment 1, it is configured such that the relation between the first explanatory variable and the objective variable is represented by a two-dimensional graph. Therefore, based on the displayed two-dimensional graph, the user can easily visually predict the variation of the objective variable to the variation of the first explanatory variable.

In FIGS. 11 and 12, although the configuration has been described in which the number of the explanatory variables serving as variation values in one inference processing is one (1), it may be configured such that the number of the explanatory variable serving as a variation value in one inference processing may be two (2) or more. For example, in a case where the number of explanatory variables serving as a variation value is set to 2, by continuously varying each of the two explanatory variables at predetermined increments and using a trained model, an objective variable corresponding to each value of the two explanatory variables is predicted. As the inference results, it is possible to display a three-dimensional graph in which a first explanatory variable between the above-described two explanatory variables is represented by the X-axis, the second explanatory variable is represented by the Y-axis, and the objective variable to be displayed is represented by the Z-axis.

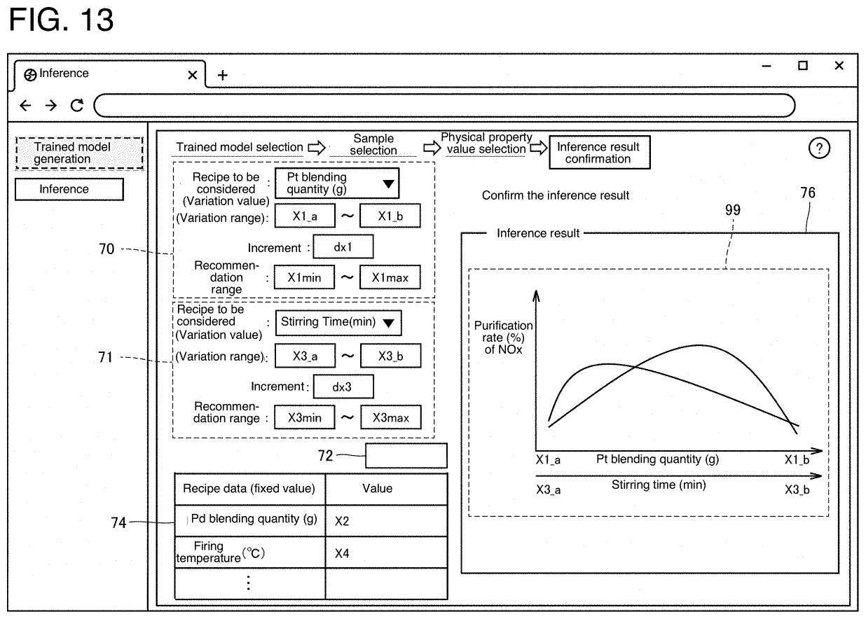

Further, in FIG. 12, a configuration has been described in which two graphs 90 and 94 are displayed on the display unit 2, corresponding to two explanatory variables in a case where the number of explanatory variables serving as variation values is two, but it may be configured such that these two graphs are displayed in a superimposed manner. FIG. 13 is a diagram schematically illustrating a third display example of inference results in the display unit 2. The display example of FIG. 13 differs from the display example of FIG. 12 in the display method of the inference results. In the display example of FIG. 13, in the display area 76, a graph 99 is displayed in which the relation between two explanatory variables serving as variation values and objective variables serving as a displayed target is shown. This graph 99 is equivalent to a graph obtained by superimposing the graph 90 of FIG. 12 on the graph 94. With this configuration, the user can relatively evaluate the influence of two explanatory variables on one objective variable based on a single graph 99.

In FIG. 13, two-dimensional graphs 90 and 94 are superimposed, but three-dimensional graphs may be superimposed. With this, the user can relatively evaluate the influence of four explanatory variables on one objective variable, based on a single graph.

### Embodiment 2

As in the second display example shown in FIG. 12, in a case where the number of the explanatory variables serving as variation values is plural, the influence of the explanatory variables on the objective variables varies depending on the type of the explanatory variable. In Embodiment 2, a configuration will be described in which influences on the objective variable among a plurality of explanatory variables serving as variation values are compared, and the type of the explanatory variable having a larger influence is stored.

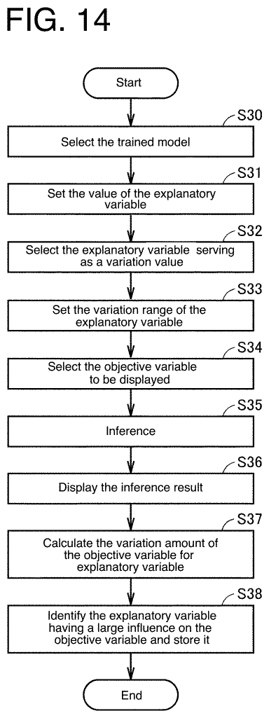

FIG. 14 is a flowchart for explaining a processing procedure of inference processing (S05 to S07 in FIG. 4) in the data processing apparatus according to Embodiment 2. The flowchart shown in FIG. 14 is a flowchart in which S37 and S38 are added to the flowchart shown in FIG. 10.

Referring to FIG. 14, when inference processing is executed by S30 to S36 which are the same as in FIG. 10, and inference results obtained by the inference processing are displayed on the display unit 2, a variation amount of the objective variable with respect to the variation of the explanatory variable is calculated by S37 for each of the plurality of explanatory variables serving variation values. The variation amount of the objective variable corresponds to the absolute value of the difference between the maximum value and the minimum value of the objective variable when the explanatory variable is continuously varied within the variation range set in S32.

In S38, based on the variation amount of the objective variable calculated in S37, an explanatory variable having a large influence on an objective variable is identified.

In S38, it may be configured such that the user identifies an explanatory variable having a large influence on a target variable by comparing a plurality of graphs displayed in the display area 76 of the display unit 2. Alternatively, it may be configured such that the data processing apparatus 1 identifies, among a plurality of variation values serving as explanatory variables, an explanatory variable having the largest objective variable variation as an explanatory variable having a large influence on an objective variable.

The type of the explanatory variable having a large influence on an objective variable identified in S38 is stored in the database 15 in association with the type of the corresponding objective variable and the information on the trained model used in the inference processing. The name of the project to which the trained model is applied is included in the information on the trained model. In a case where the sample is a three-way catalyst, the name of the project is, for example, the “improvement of a purification performance of a three-way catalyst.” “improvement of a heat resistance of a three-way catalyst,” and the like.

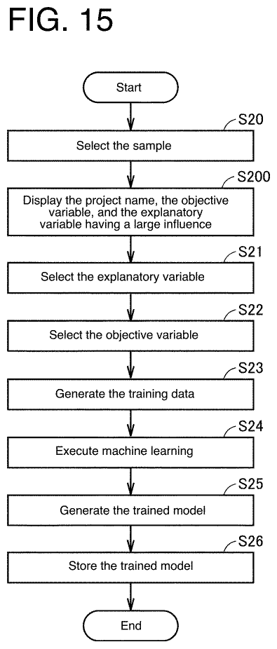

The information stored in the database 15 in S38 of FIG. 14 can be used in the training phase. FIG. 15 is a flowchart for describing the processing procedure of generating training data (S02 in FIG. 4), machine learning (S03 in FIG. 4), and storing the trained model (S04 in FIG. 4) in the data processing apparatus according to Embodiment 2. The flowchart shown in FIG. 15 is a flowchart in which S200 is added to the flowchart shown in FIG. 7.

Referring to FIG. 15, in S20 which is the same as in FIG. 7, a UI screen (sample selection screen) is displayed on the display unit 2. The user can select a sample to be used for generating training data by operating the sample selection screen using the operation unit 3. When the sample selection is completed, selection sample extraction data is generated.

By S200, the display unit 2 displays the information stored in the database 15 in S38 of FIG. 14 in the past inference processing. Specifically, the information (name of the project) regarding the trained model used in the past inference processing and the information regarding the objective variable predicted from the trained model, and the type of the explanatory variable having a large influence on the objective variable are displayed on the display unit 2.

Next, an explanatory variable and an objective variable to be used for generating training data are selected by S21 and S22 which are the same as those in FIG. 7. On the display unit 2, UI screens (explanatory variable selection screen and objective variable selection screen) are displayed. As described above, the display unit 2 shows the information regarding the name of the project to which the trained model is applied, the type of the objective variable predicted from the trained model, and the type of the explanatory variable having a large influence on the objective variable. Therefore, while referring to the information, the user can select the explanatory variable and the objective variable that constitute training data, depending on the project to which a trained model to be newly generated is applied. For example, the user can select the objective variable and the explanatory variable so as to include the objective variable associated with the trained model with the same project and the explanatory variable having a large influence on the objective variable.

As described above, according to the data processing apparatus of Embodiment 2, a trained model can be generated using the training data including the explanatory variable having a large influence on the objective variable. This can enhance the usefulness of the trained model for the project.

### Embodiment 3

In the above-described Embodiment 1, a configuration has been described in which in the inference phase, the explanatory variable serving as a variation value among the plurality of explanatory variables to be given to the trained model is selected based on the user inputs to the GUIs 70 and 71 (see FIGS. 11 and 12).

According to the above-described configuration, as the inference result, for the explanatory variable selected by the user, a graph showing the relation between the explanatory variable and the objective variable can be displayed on the display unit 2. On the other hand, as for which explanatory variable among the plurality of explanatory variables is to be selected, it depends on the user experience and the skill level. Therefore, even in the case of an explanatory variable in which the influence on an objective variable is large, a graph showing the relation between the explanatory variable and the object variable cannot be displayed unless the user selects it as a variation value. Consequently, there is a concern that the user fails to consider a critical explanatory variable.

Therefore, in Embodiment 3, a configuration for displaying an inference result about an explanatory variable having a large influence on an objective variable will be described. Note that the operation of the data processing apparatus according to Embodiment 3 is basically the same as the operation of the data processing apparatus according to the above-described Embodiment 1, except for the inference processing described below.

### (1) First Configuration Example

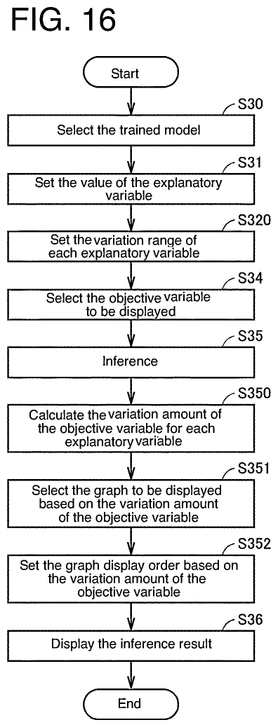

FIG. 16 is a flowchart for explaining inference processing in a data processing apparatus according to a first configuration example of Embodiment 3. The flowchart shown in FIG. 16 is a flowchart in which S32 is replaced with S320, and S350 to S352 are added with respect to the flowchart shown in FIG. 10.

Referring to FIG. 16, in S30 and S31, which are the same as those of FIG. 10, when a trained model to be used for inference processing is selected and values of a plurality of explanatory variables to be inputted to the trained model are set, in S320, the variation range is set for each of the plurality of explanatory variables. That is, in this first configuration example, it differs from the above-described embodiment in that the plurality of explanatory variables to be inputted to the trained model are all set as variation values.

The variation range of each explanatory variable can be set based on the training data used to generate the trained model. For example, the data corresponding to each explanatory variable can be extracted from the training data, the minimum value of the extracted data can be set to the lower limit value of the variation range, and the maximum value of the data can be set to the upper limit value of the variation range.

In S34, which is the same as that in FIG. 10, an objective variable to be displayed is selected from objective variables predicted from the trained model. The user can select the objective variable to be displayed by operating the user interface screen displayed on the display unit 2 using the operation unit 3.

In S35, which is the same as that in FIG. 10, an objective variable is predicted by inputting a plurality of explanatory variables to the trained model selected in S30. In this inference processing, one explanatory variable among the plurality of explanatory variables is continuously varied at predetermined increments, and an objective variable corresponding to each value of the explanatory variable is predicted.

Note that when the one explanatory variable is varied, the values of explanatory variables other than the explanatory variable are set as fixed values. The values of other explanatory variables are fixed as the values set in S31. The values are based on the recipe data, the physical property data, the analysis data, and the feature of the sample to be analyzed.

When a variation of an objective variable for a variation of one explanatory variable is predicted, another explanatory variable is varied to predict a variation of an objective variable. When the variation of the objective variable is predicted for all of the plurality of explanatory variables, the inference processing of S35 is terminated.

When the inference processing of S35 is completed, a graph to be displayed as an inference result is selected from the inference results of the plurality of objective variables respectively corresponding to the plurality of explanatory variables. Specifically, first, in S350, for each of a plurality of explanatory variables, a variation amount of an objective variable to the variation of the explanatory variable is calculated. The variation amount of the objective variable corresponds to the absolute value of the difference between the maximum value and the minimum value of the objective variable when the explanatory variable is continuously varied within the variation range set in S320.

Next, in S351, graphs to be displayed are selected based on the variation amount of the objective variables calculated in S350. In S351, graphs showing the relation between the explanatory variable and the objective variable are selected in descending order of the variation amount of the objective variable among the plurality of inference results. The number of graphs to be selected as display targets can be set in advance by the user. For example, a predetermined number of graphs counting from the largest variation amount of the objective variable can be displayed. Alternatively, graphs in which the variation amount of the objective variable is greater than or equal to a predetermined value may be displayed.

In S352, for the selected graphs to be displayed in S351, the display order is set. Specifically, the display order is set such that graphs are arranged in descending order of the variation amount of the objective variable with the largest variation amount displayed first.

In S36, the inference results acquired by the inference processing in S35 are displayed on the display unit 2. On the display unit 2, the selected graphs are displayed according to the display order set in S352.

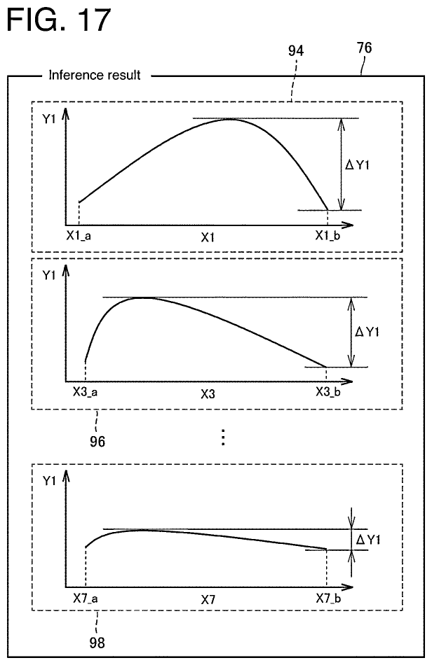

FIG. 17 is a diagram schematically illustrating a display example of inference results in the first configuration example. In FIG. 17, the display area 76 displaying the inference results is extracted from the display unit 2 shown in FIG. 11.

In the example of FIG. 17, in the display area 76 of the display unit 2, a plurality of graphs 94, 96, and 98 indicating the relation between the explanatory variable serving as a variation value and the objective variable to be displayed is displayed. The graph 94 is a two-dimensional graph in which the explanatory variable X1 is represented by the the horizontal axis, and the objective variable Y1 is represented by the vertical axis. The graph 96 is a two-dimensional graph in which the explanatory variable X3 is represented by the horizontal axis, and the objective variable Y1 is represented by the vertical axis. The graph 98 is a two-dimensional graph in which the explanatory variable X7 is represented by the horizontal axis, and the objective variable Y1 is represented by the vertical axis.

In each of the graphs 94, 96, and 98, ΔY1 represents a variation amount of the objective variable Y1 when the corresponding explanatory variable is continuously varied within a variation range. The graph 94 has the largest variation amount ΔY1, and the graph 96 has the second largest variation amount ΔY1. The graph 98 has the smallest variation amount ΔY1. That is, in the display area 76, a plurality of graphs 94, 96, and 98 is displayed side by side in descending order of the variation amount ΔY1 of the objective variable Y1.

Note that as described above, the number of graphs to be displayed in the display area 76 can be set in advance by the user. For example, in a case where the number of graphs to be displayed in the display area 76 is set to N (N≥1), a total of N pieces of graphs are displayed side by side in the display area 76 in descending order of the variation amount ΔY1 of the objective variable Y1.

Alternatively, it may be configured such that graphs in which the variation amount ΔY1 of the objective variable Y1 is equal to or larger than a predetermined value are displayed in the display area 76. In this case, the graphs in which the variation amount ΔY1 of the objective variable Y1 is equal to or larger than the predetermined value are displayed side by side in the display area 76 in descending order of the variation amount ΔY1.

As described above, according to the first configuration example, among the plurality of explanatory variables to be given to the trained model, one having a larger variation amount of the objective variable with respect to the variation of the explanatory variable is preferentially selected, and a graph showing the relation between the selected explanatory variable and the objective variable is displayed on the display unit 2. According to this, regardless of the user's experience and skill level, explanatory variables having a larger influence on an objective variable are automatically selected, and the inference result of the variation of the objective variable with respect to the variation of the explanatory variable is displayed. This can reduce the possibility that a user fails to consider critical explanatory variables.

Further, in the display unit 2, graphs are displayed in the order that the variation amount of the objective variable with respect to the variation of the explanatory variable is large, and therefore, the graph can be effectively displayed for the explanatory variable having a larger influence on the objective variable. Therefore, the usefulness of the inference result can be improved.

### (2) Second Configuration Example

In the above-described first configuration example, all of the plurality of explanatory variables to be given to the trained model are set to variation values, and therefore, there is a concern that the computational amount required for the inference processing increases as the number of explanatory variables increases. Therefore, in a second configuration example and a third configuration example which will be described later, a configuration is described in which the data processing apparatus 1 automatically selects explanatory variables serving as variation values prior to executing the inference processing.

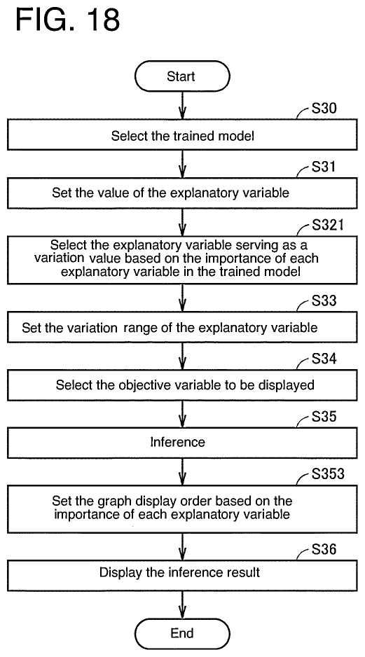

FIG. 18 is a flowchart for explaining the inference processing in the data processing apparatus according to a second configuration example of Embodiment 3. The flowchart shown in FIG. 18 is a flowchart in which S32 is replaced with S321, and S353 is added to the flowchart shown in FIG. 10.

Referring to FIG. 18, in S30 and S31, which are the same as those of FIG. 10, when a trained model to be used for the inference processing is selected, and values of the plurality of explanatory variables to be inputted to the trained model are set, the inference unit 32 of the data processing apparatus 1 acquires the importance of each explanatory variable to be input to the trained model in S321.

The importance of each explanatory variable is a quantification of how much the corresponding explanatory variable has contributed to the performance of the model. Specifically, the importance of each explanatory variable can be calculated by applying a decision tree algorithm to a plurality of explanatory variables. Note that as the decision tree algorithm, any known algorithm can be adopted, but, for example, a random forest or the like can be used.

The inference unit 32 selects an explanatory variable serving as a variation value based on the importance of each explanatory variable. Specifically, the inference unit 32 selects an explanatory variable with higher importance as an explanatory variable preferentially serving as a variation value. The number of explanatory variables serving as variation values can be set in advance by the user. For example, a predetermined number of explanatory variables counting from the highest importance may be set as a variation value. Alternatively, an explanatory variable in which the importance is equal to or greater than a predetermined value may be set as a variation value.

When an explanatory variable serving as a variation value is selected in S321, the inference unit 32 sets the variation range of each explanatory variable in S33. The variation range of each explanatory variable may be set based on the training data used to generate each trained model. For example, data corresponding to each explanatory variable may be extracted from the training data, the minimum value of the extracted data may be set to the lower limit value of the variation range, and the maximum value of data may be set to the upper limit value of the variation range.

In S34, which is the same as that in FIG. 10, an objective variable to be displayed is selected from the objective variable predicted from the trained model. The user can select the objective variable to be displayed by operating the user interface screen displayed on the display unit 2 using the operation unit 3.

In S35, which is the same as that in FIG. 10, an objective variable is predicted by inputting the explanatory variables set in S31 and S321 to the trained model selected in S30. In this inference processing, the inference unit 32 continuously varies the one explanatory variable among the explanatory variables serving as variation values at predetermined increments and predicts an objective variable corresponding to each value of the explanatory variable.

Note that in a case where the one explanatory variable is varied, the values of explanatory variables other than the explanatory variable are set as fixed values. For example, the values of the other explanatory variables are fixed as the values set in S31. The values are based on the recipe data, the physical property data, the analysis data, and the feature of the sample to be analyzed. When a variation of an objective variable with respect to a variation of one explanatory variable is predicted, the inference unit 32 varies another explanatory variable to predict the variation of the objective variable. When the variation of the objective variable is predicted for all of explanatory variables serving as variation values, the inference processing of S35 is terminated.

When the inference processing in S35 is completed, in S353, the display data generation unit 34 sets the display order of the graphs to be displayed as the inference results based on the importance of each explanatory variable. Specifically, the graph display order is set such that graphs are arranged in descending order from the higher importance of the explanatory variable with the graph showing the inference result for the highest explanatory variable arranged first.

In S36, the display data generation unit 34 displays the inference results acquired by the inference processing in S35 on the display unit 2. On the display unit 2, the graphs selected to be displayed are displayed according to the display order set in S352.

As described above, according to the second configuration example, an explanatory variable having higher importance in a trained model among a plurality of explanatory variables to be given to the trained model is selected as a variation value, and graphs showing the relation between the selected explanatory variable and the objective variable are displayed on the display unit 2. This displays the inference results of the variation of the objective variable to the variation of the explanatory variable having a larger influence on the objective variable, regardless of the user's experience or skill level. This can reduce the possibility that a user fails to consider a critical explanatory variable.

Further, in the display unit 2, graphs are displayed in descending order of the higher degree of influence of the explanatory variable with respect to the objective variable, and therefore, it is possible to effectively display a graph of an explanatory variable having the higher degree of influence on the objective variable. Therefore, the usefulness of the inference result can be improved.

### (3) Third Configuration Example

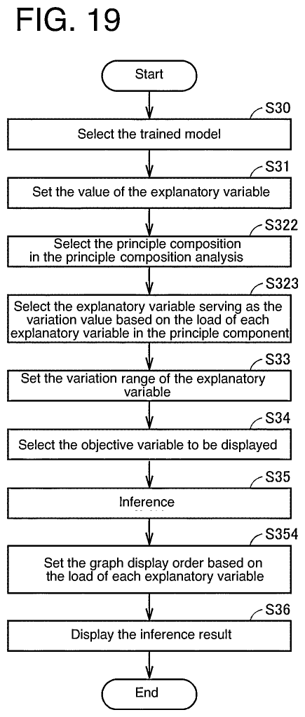

FIG. 19 is a flowchart for explaining inference processing in a data processing apparatus according to a third configuration example of Embodiment 3. The flowchart shown in FIG. 19 is a flowchart in which S32 is replaced with S322 and S323, and S354 is added to the flowchart shown in FIG. 10.

Referring to FIG. 19, in S30 and S31, which are the same as those of FIG. 10, when a trained model to be used for the inference processing is selected, and values of a plurality of explanatory variables to be inputted to the trained model are set, the inference unit 32 selects an explanatory variable as a variation value from a plurality of explanatory variables. In the third configuration example, an explanatory variable serving as a variation value is selected by utilizing a principal component analysis executed for the plurality of explanatory variables.

The principal component analysis is generally implemented to reduce the dimensionality of data, as the preprocessing to a large amount of data. By implementing the principal component analysis, a plurality of explanatory variables is aggregated into a smaller number of synthesis variables (principal component). The result of the principal component analysis is acquired as a principal component score, which is the transformed value corresponding to the original explanatory variable, and a principal component load corresponding to the weight of the explanatory variable for each principal component score.

In S322, one principal component is selected from a predetermined number of principal components acquired by the principal component analysis. For example, the inference unit 32 may select one principal component according to the user input. In this case, the user can select one principal component based on the contribution rate of each principal component. Note that the contribution rate of the principal component is obtained by dividing the eigenvalues of each principal component by the sum and indicates how much each principal components occupies in the entirety. Note that the inference unit 32 may be configured to select a first principal component having the highest contribution rate regardless of the user input.

In S323, the inference unit 32 selects an explanatory variable serving as a variation value based on the weight (principal component load) of each explanatory variable in one principal component selected in S322.

Specifically, the i^{th} principal component z is a combination of the original p pieces of variables X1, X2, . . . , Xp multiplied by the weight w (principal component load), and can be expressed by the following formula. Note that the sum of squares of p pieces of wj (j=1, 2, . . . , p) is 1.

z=w1X1+w2X2+ . . . +wpXp

In the above-described formula, the larger the absolute value of the weight w (principal component load), the higher the contribution of the corresponding explanatory variable X to the principal component z, i.e., it can be said that the explanatory variable is an explanatory variable characterizing the principal component. Therefore, in S323, the inference unit 32 selects an explanatory variable having a larger weight (principal component load) as an explanatory variable preferentially serving as a variation value.

Note that the number of explanatory variables serving as variation values can be set in advance by the user. For example, a predetermined number of explanatory variables counting from the highest weight (principal component load) may be variation values. Alternatively, an explanatory variable in which the weight (principal component load) is equal to or greater than a predetermined value may be a variation value.

When the explanatory variable serving as a variation value is selected in S323, the inference unit 32 sets the variation range of each explanatory variable in S33. The variation range of each explanatory variable can be set based on the training data used to generate each trained model. For example, data corresponding to each explanatory variable can be extracted from the training data, the minimum value of the extracted data can be set to the lower limit value of the variation range, and the maximum value of data can be set to the upper limit value of the variation range.

In S34, which is the same as that in FIG. 10, an objective variable to be displayed is selected from the objective variables predicted from a trained model. The user can select the objective variable to be displayed by operating the user interface screen displayed on the display unit 2 using the operation unit 3.

In S35, which is the same as that in FIG. 10, the inference unit 32 predicts an objective variable by inputting an explanatory variable set in S31 and S321 to the trained model selected in S30. In this inference processing, one explanatory variable among variation values as explanatory variables is continuously varied at predetermined increments, and an objective variable corresponding to each value of the explanatory variable is predicted. Note that when the one explanatory variable is varied, the values of explanatory variables other than the explanatory variable are set as fixed values. For example, the value of the other explanatory variable is fixed to the value set in S31. When a variation of an objective variable with respect to a variation of one explanatory variable is predicted, the inference unit 32 varies the one explanatory variable to predict a variation of an objective variable. When the variation of the objective variable is predicted for all of explanatory variables serving as variation values, the inference processing of S35 is terminated.

When the inference processing in S35 is completed, the display data generation unit 34 sets the display order of graphs to be displayed as inference results, based on the weights (principal component load) of each explanatory variable in S353. Specifically, the display order of the graphs is set such that the graph indicating the inference result for the explanatory variable having the highest weight (principal component load) is set as the first graph and the graphs are arranged in descending order from the higher weight (principal component load).

In S36, the display data generation unit 34 displays the inference results acquired by the inference processing in S35 on the display unit 2. In the display unit 2, the selected graphs are displayed according to the display order set in S354.

As described above, according to the third configuration example, explanatory variables having larger principal component loads for a particular principal component among a plurality of explanatory variables to be given to the trained model are selected as variation values, and graphs showing the relation between the selected explanatory variable and the objective variable are displayed on the display unit 2. With this, the inference result of variables of the objective variables to the variable of the explanatory variable with higher contribution to the principal component, regardless of the user's experience and skill. This can reduce the possibility that a user fails to consider critical explanatory variables.

Further, the graphs are displayed in descending order of the principal component load of the explanatory variable on the display unit 2, and therefore, it is possible to display the graphs having an explanatory variable higher contribution to the principle component. Therefore, the usefulness of the inference result can be improved.

### Embodiment 4

In the training phase, supervised learning is performed in which an explanatory variable of the generated training data is used as an input of the learning model, and an objective variable of the training data is used as the ground truth data of the output of the learning model. In Embodiment 4, a configuration in which a user can select a learning model will be described. Note that the operation of the data processing apparatus according to Embodiment 4 is basically the same as the operation of the data processing apparatus according to Embodiment 1 described above, except for the learning processing described below.

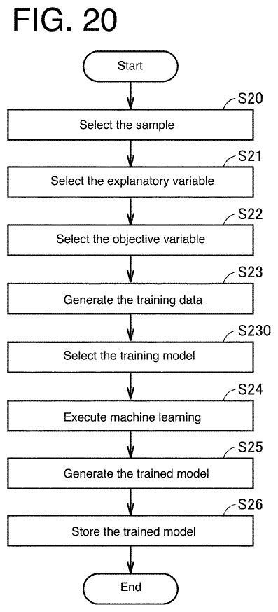

FIG. 20 is a flowchart for explaining the processing procedure of generating training data (S02 in FIG. 4), machine learning (S03 in FIG. 4), and storing the trained model (S04 in FIG. 4) in the data processing apparatus according to Embodiment 4. The flowchart shown in FIG. 20 is a flowchart in which S230 is added to the flowchart shown in FIG. 7.

Referring to FIG. 20, when training data is generated by S20 to S23 which are the same as in FIG. 7, the learning model to be used for machine learning is selected by S230. On the display unit 2, a UI screen (model selection screen) is displayed. The model selection screen is a UI screen for the user to select the learning model. In a model selection window, a plurality of learning models is displayed in a listed manner. The plurality of learning models is, for example, a polynomial regression model, and the orders of the polynomials are different from each other. In addition, the user can add an interaction term between terms, logarithmic and exponential terms.

In machine learning, as the order of the polynomial is increased, the accuracy with respect to the learning data is enhanced, but it sometimes becomes a so-called “overfitting” in which the accuracy with respect to the unknown data is decreased.

In Embodiment 4, as the order of the learned model is increased, or as the model is complicated by including the interaction term, logarithmic, and exponent, the complex relation between the explanatory variable and the objective variable constituting the training data can be expressed. On the other hand, by simplifying the learning model, the above-described overfitting can be avoided. In S230 of FIG. 20, the user can select the order of the learning model after comparing these merits and demerits, and therefore, the optimum machine learning can be performed.

### Embodiment 5

In Embodiment 1 described above, a sample list (FIG. 6) is generated based on the data stored in the database 15 (S01 in FIG. 4 and FIG. 5). At this time, by analyzing the analysis data of the sample using dedicated data analysis software, the features of the sample can be extracted (S13 in FIG. 5). The features includes a peak area with respect to a predetermined mass number acquired by analyzing the chromatogram acquired by a GC-MS, an abundance ratio of a predetermined substance acquired by analyzing the NMR spectrum acquired by the NMR, a particle diameter of a particle present in the three-way catalyst acquired by analyzing the SEM image, a mean particle diameter, and a particle diameter of a particle present in the three-way catalyst, acquired by analyzing the TEM image acquired by the TEM.

In the processing of extracting these features, by changing the condition for processing analytical data or by changing the condition for calculating the feature, even in the case of analysis data of the same sample, the features to be extracted may sometimes become different. In this case, since the training data generated from the sample list varies depending on the processing conditions of the analysis data, the trained model generated from the training data also varies depending on the processing conditions of the analytical data and/or the calculation conditions of the feature. When the trained models are different, in the inference processing, even if the explanatory variable given to the trained model is the same, the predicted objective variable may be different. Therefore, the influence of the explanatory variable on the objective variable derived from the inference result may also differ depending on the difference in the trained model.

In Embodiment 5, a configuration for acquiring data process conditions suitable for considering the influence of the explanatory variable on the objective variable will be described. Hereinafter, the processing performed by the data processing apparatus according to Embodiment 5 in each of the training phase and the inference phase will be described.

### <Training Phase>

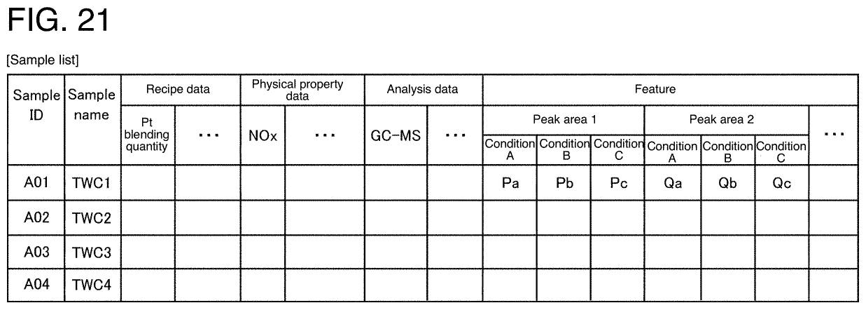

FIG. 21 is a diagram showing a configuration example of a sample list. FIG. 21 shows a configuration example of a sample list in a case where the sample is a three-way catalyst. The sample list shown in FIG. 21 differs from the sample list shown in FIG. 6 in that it includes a plurality of features extracted by processing the analysis data of the sample under a plurality of processing conditions.

In the example of FIG. 21, the feature includes a peak area 1 to a first mass number and a peak area 2 to a second mass number, acquired by analyzing the chromatogram acquired by a GC-MS.

The peak area 1 is composed of three values Pa, Pb, and Pc that differ from each other in the data processing condition (method for calculating the peak area). Pa is the peak area 1 calculated using the processing condition A, Pb is the peak area 1 calculated using the processing condition B, and Pc is the peak area 1 calculated using the processing condition C. The peak area 2 is composed of three values Qa, Qb, and Qc that differ from each other in the data processing condition (method for calculating the peak area). Qa is the peak area 2 calculated using the processing condition A, Qb is the peak area 2 calculated using the processing condition B, and Qc is the peak area 2 calculated using the processing condition C.

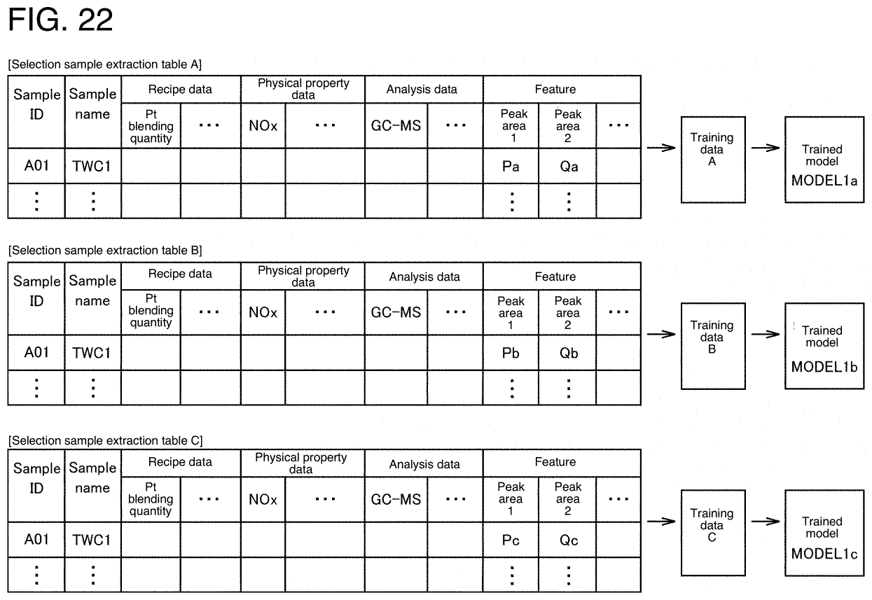

In the process of generating the training data (S02 in FIG. 4), when the sample used for generating the training data is selected (S20 in FIG. 7), data included in the row of the selected sample is extracted from the sample list shown in FIG. 21, and the selection sample extraction table is generated. FIG. 22 is a diagram illustrating a configuration example of a selection sample extraction table. Three types of selection sample extraction tables are shown in FIG. 22.

The selection sample extraction table A is configured to include the peak area 1 and the peak area 2, which are features acquired using the processing condition A. The selection sample extraction table B is configured to include the peak area 1 and the peak area 2, which are features acquired using the processing condition B. The selection sample extraction table C is configured to include the peak area 1 and the peak area 2, which are features acquired using the processing condition C.

That is, the selection sample extraction tables A to C are generated from the analysis data of the same sample, but the data processing conditions for extracting the feature from the analysis data are different from each other. Consequently, the selection sample extraction tables A to C have the same type of data but differ from each other in the data value.

When the explanatory variable and the objective variable to be used for generating the training data are selected (S21, S22 in FIG. 7), three types of training data tables are generated by extracting the data matching the explanatory variable and the data matching the objective variable, from each of the selection sample extraction tables A to C. To each of the three types of training data tables, for each sample, the data matching the explanatory variable and the data matching the objective variable matching are inputted. Based on the three types of generated training data tables, training data A to C is generated.

Three types of trained models are generated by performing supervised training using the training data A to C. The trained model MODEL1a is a trained model generated by machine learning using the training data A. The trained model MODEL1b is a trained model generated by machine learning using the training data B. The trained model MODEL1c is a trained model generated by machine learning using the training data C.

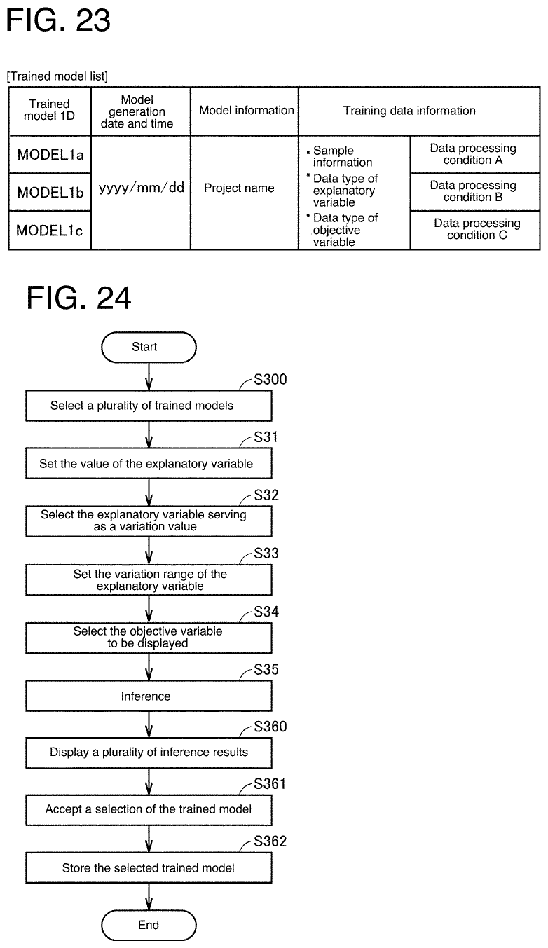

The generated trained models MODEL1a, MODEL1b, and MODEL1c are registered in the trained model list stored in the database 15. FIG. 23 is a diagram showing a configuration example of a trained model list. The trained model list shown in FIG. 23 differs from the trained model list shown in FIG. 9 in that identification information for identifying the training data includes data processing conditions used to extract the feature from the analysis data.

The trained models MODEL1a, MODEL1b, and MODEL1c are common in the name of the project to which the trained model is applied, the sample information used to generate the training data, and the type of data selected for the explanatory variable and the objective variable. On the other hand, the processing conditions of the analysis data (for example, the method for calculating the peak area of the chromatogram) used to generate the data are different from each other.

### <Inference Phase>

FIG. 24 is a flowchart for explaining the processing procedure of the inference processing (S05 to S07 in FIG. 4) in the data processing apparatus according to Embodiment 5. The flowchart shown in FIG. 24 is a flowchart in which S30 in the flowchart shown in FIG. 10 is replaced with S300, and S36 is replaced with S360 to S362.

Referring to FIG. 24, in S300, a plurality of trained models to be used for inference processing are selected. The display unit 2 displays a UI screen (trained model selection screen) generated based on the trained model list (FIG. 23) stored in the database 15. By operating the UI screen using the operation unit 3, the user can select a plurality of trained models in which only the processing conditions of the analysis data are mutually different. Hereinafter, it is assumed that the trained models MODEL1a, MODEL1b, and MODEL1c are selected.

In S31 which is the same as in FIG. 10, the value of the explanatory variable to be inputted to each trained model is set. In S32, “explanatory variable serving as a variation value” is selected from the plurality of explanatory variables. In S33, the variation range is set for the explanatory variable serving as a variation value. In S34, out of the objective variables predicted from the respective trained models, an objective variable to be displayed is selected.

In S35 which is the same as in FIG. 10, an objective variable is predicted by inputting explanatory variables set in S31 to S33 to each of the plurality of trained models selected in S300. In this inference processing, for each trained model, it is predicted how the objective variable varies, corresponding to the respective values of some continuously varying explanatory variables of the plurality of explanatory variables.

In S360, a plurality of inference results acquired by the inference processing in S35 is displayed on the display unit 2. In the display unit 2, a graph indicating a variation with respect to a variation of some of explanatory variables is displayed for the objective variable selected as the display target corresponding to the plurality of trained models.

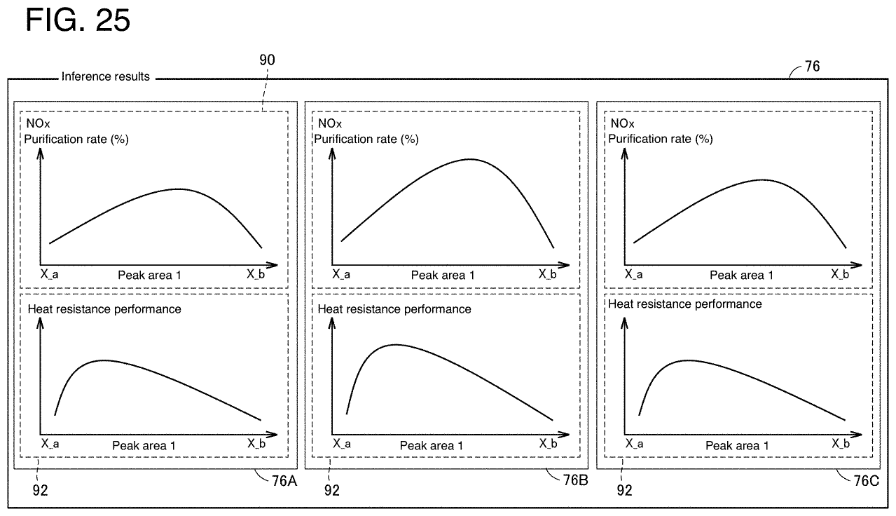

FIG. 25 is a diagram schematically illustrating display examples of a plurality of inference results in the display unit 2. In FIG. 25, the display area 76 displaying the inference result of the display unit 2 is extracted and shown.

The display area 76 is provided with a display area 76A in which the inference result of the inference processing using the trained model MODEL1a is displayed, a display area 76B in which the inference result of the inference processing using the trained model MODEL1b is displayed, and a display area 76C in which the inference result of the inference processing using the trained model MODEL1c is displayed.

In each of the display areas 76A, 76B, and 76C, graphs 90 and 92 showing the relation between the explanatory variable serving as a variation value and the objective variable to be displayed are displayed. In the example of FIG. 25, “peak area 1” is selected as the explanatory variable serving as a variation value, and “NOx-purification rate (%)” and “heat-resistant performance” are selected as the objective variables to be displayed. The graphs 90 and 92 show how the NOx purification rate (%) and the heat resistance performance vary when the peak area 1 is continuously varied within a predetermined variation range.

Comparing the graphs 90 between the display areas 76A, 76B, and 76C, the difference in the trained model indicates that the influence of the explanatory variable on the objective variable is different even if the sample to be analyzed is the same. The same can be applied to the graph 92.

By comparing the graphs 90, 92 displayed in these three display areas 76A, 76B, and 76C, the user can select a trained model that is considered to be suitable for considering the relation between the explanatory variable and the objective variable.

Note that in FIG. 25, an example is shown in which three display areas 76A, 76B, and 76C are displayed side by side, but it may be configured such that the display areas 76A, 76B, and 76C are switched and displayed in accordance with the user's operation in a state in which only one display area is displayed.

Returning to FIG. 24, in S361, an appropriate trained model is selected by the user. For example, a UI screen for selecting an appropriate trained model is displayed on the display unit 2. The user can select an appropriate trained model by operating the UI screen using the operation unit 3. In S362, the information on the appropriately trained model selected by the user is stored in the database 15. The information on the appropriately trained model includes the name of the project to which the trained model is applied, the sample information used to generate the training data, the type of data selected as the explanatory variable and the objective variable, and the processing condition of the analytical data used to extract the data.

In this way, by storing the information on the appropriately trained model selected based on the inference result in the database 15, in future, in a situation of generating a trained model using a sample similar to the sample analyzed in this inference processing, it is possible to read out the data processing conditions used to generate an appropriately trained model from the database 15 and present it to the user. Note that the “sample similar to the sample” means that at least one of recipe data, physical property data, and analytical data of a sample is the same or similar.

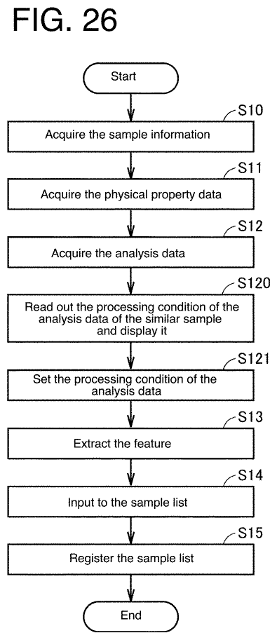

FIG. 26 is a flowchart for explaining the processing procedure of generating a sample list (S01 in FIG. 4). The flowchart shown in FIG. 26 is a flowchart in which S120 and S121 are added to the flowchart shown in FIG. 5.

When the sample information, the sample's physical property data, and the sample's analytical data are acquired in S10 to S12 which are the same as those in FIG. 5, in S120, the processing condition of the analysis data in the sample similar to the sample of this time is read out from the database 15 and displayed in the display unit 2, by referring to the data. The processing condition of the analysis data read out from the database 15 is included in the information on the trained model that the user previously determined to be appropriate to consider the relation between the explanatory variable and the objective variable.

In S121, the processing condition of the analytical data acquired in S12 is set via the operation unit 3. In Step 13 which is the same as in FIG. 5, the analysis data acquired in Step S12 is processed using the processing condition set in S121 to extract the feature of the sample. In S14, the obtained sample information, physical property data of the sample, and analysis data, and feature of the sample are inputted to the sample list (FIG. 6). In S15, the sample list is registered in the database 15 by assigning the identification information of the sample list.

In this way, in the training phase, using the data processing conditions determined to be suitable for considering the relation between the explanatory variable and the objective variable, the features are extracted from the analysis data of the sample, and a sample list is generated. And, a trained model is generated using the training data generated based on the sample list. With this, in the inference phase, the relation between the explanatory variable given to the trained model and the objective variable predicted from the trained model becomes a relation determined to be appropriate by the user. Therefore, the usefulness of the inference results can be enhanced.

### Other Configuration Examples

(1) In the above-described embodiment, a configuration example of a UI screen for receiving user's operations when inference processing is executed and a configuration for displaying the inference results (see FIGS. 11 and 12) on the display unit 2 connected to the data processing apparatus 1 have been described. However, in place of the display unit 2, for example, it may be configured such that an information terminal, such as, e.g., a desktop personal computer (PC), a notebook PC, and a portable terminal (tablet terminal, smartphone), is connected to the data processing apparatus 1 to display UI screens and inference results on the information terminal.

(3) In the above-described embodiment, a configuration example has been described in which the type of data of explanatory variables to be inputted to a trained model and the type of data of objective variables to be predicted by the trained model are automatically determined by selecting the trained model to be used for the inference processing. However, it may be configured such that the trained model to be used for the inference processing is automatically determined by selecting the type of data of explanatory variables to be inputted to the trained model and the type of data of objective variables to be predicted. In this case, the user can select the type of data of the explanatory variable and the type of data of the objective variable by checking the selection icon on the UI screen (objective variable selection screen and explanatory variable selection screen) displayed on the display unit 2 with the operation unit 3. The Inference unit 32 refers to a trained list (FIG. 9) stored in the database 15, and can determine the trained model associated with the training data including the type of data of the selected explanatory variables and the type of data of objective variables as a trained model to be used in the estimation processing.

(4) In the above-described embodiment, the configuration is exemplified in which the data processing apparatus 1 is provided with the training unit 30 and the inference unit 32 (see FIG. 3), but the training unit 30 and the inference unit 32 may be separately provided.

[Aspects] It will be understood by those skilled in the art that the plurality of exemplary embodiments described above is illustrative of the following aspects.

(Item 1) A data processing apparatus according to a first aspect is provided with: an inference unit configured to predict an objective variable from a plurality of explanatory variables by using a trained model; and a display data generation unit configured to generate data for displaying an inference result by the inference unit. The inference unit is configured to set a first explanatory variable selected from the plurality of explanatory variables as a variation value and set second explanatory variables other than the first explanatory variable as fixed values. The inference unit predicts, by using the trained model, the objective variable when the first explanatory variable is continuously varied within a predetermined variation range. The display data generation unit generates data indicating a variation of the objective variable with respect to a variation of the first explanatory variable.

According to the data processing apparatus recited in the above-described Item 1, the user can easily predict how the objective variable varies when the first explanatory variable serving as a variation value is continuously varied, based on the displayed data. Therefore, the usefulness of the inference result can be enhanced.

(Item 2) In the data processing apparatus as recited in the above-described Item 1, the display data generation unit generates a two-dimensional graph in which the first explanatory variable is represented by a first axis, and the objective variable is represented by a second axis.

According to the data processing apparatus as recited in the above-described Item 2, the user can easily visually predict the variation of an objective variable with respect to the variation of the first explanatory variable, based on the displayed two-dimensional graph.

(Item 3) In the data processing apparatus as recited in the above-described Item 1 or 2, the inference unit is configured to select two or more of first explanatory variables from the plurality of explanatory variables. The inference unit predicts, by using the trained model, an objective variable when the first explanatory variable is continuously varied within a variation range, for each of the selected two or more first explanatory variables. A display unit is connected to the data processing apparatus. The display data generation unit generates two or more of two-dimensional graphs corresponding to two or more first explanatory variables. The display data generation unit displays the generated two or more of the two-dimensional graphs in a superimposed manner.

According to the data processing apparatus as recited in the above-described Item 3, it becomes possible to relatively evaluate the influence of each of the two or more of the first explanatory variables on the objective variable, based on two or more two-dimensional graphs displayed on the display unit in a superimposed manner.

(Item 4) In the data processing apparatus as recited in any one of the above-described Item 1 to 3, the display data generation unit is configured to provide a first user interface for selecting the first explanatory variable and setting the variation range. The first user interface includes information on a recommendation range of the variation range.

According to the data processing apparatus as recited in the above-described Item 4, the user's convenience in the inference processing can be improved.

(Item 4) In the data processing apparatus as recited in the above-described Item 3, the trained model is a model generated by machine learning using training data in which the plurality of explanatory variables are inputs, and the objective variable is a ground truth output. The recommendation range is set based on a value of the first explanatory variable included in the training data.

According to the data processing apparatus as recited in the above-described Item 4, it is possible to provide the user with a recommendation range in which the accuracy of the inference result is guaranteed.

(Item 5)

In the data processing apparatus as recited in the above-described Item 3, the display data generation unit is configured to further provide a second user interface for setting a value of the second explanatory variable.

According to the data processing apparatus as recited in the above-described Item 5, the user's convenience in the inference processing can be improved.

(Item 6) The data processing apparatus as recited in the above-described Item 4, the display data generation unit is further provided with: a training data generation unit configured to generate the training data; a training unit configured to generate the trained model by machine learning using the training data; and a database configured to store the trained model in association with the training data.

According to the data processing apparatus described in the above-described Item 6, by referring to the training data associated with the trained model used in the inference processing, the type of data of the explanatory variable to be inputted to the trained model and the type of data of the objective variable acquired by the trained model can be automatically determined. Alternatively, the trained model to be used for the inference processing can be automatically determined by selecting the type of data of the explanatory variable to be inputted to the trained model and the type of data of the objective variable to be acquired by the trained model.

(Item 7) In the data processing apparatus as recited in the above-described Item 1 or 2, the trained model is a model generated by machine learning using training data in which the plurality of explanatory variables are inputs, and the objective variable is a ground truth output. The inference unit selects at least one of the first explanatory variables from the plurality of explanatory variables based on the importance of each explanatory variable in the trained model. The inference unit predicts, by using the trained model, the objective variable when the first explanatory variable is continuously varied within the variation range, for each of the selected at least one of the first explanatory variables.

According to the data processing apparatus as recited in the above-described Item 7, out of the plurality of explanatory variables to be given to the trained model, an explanatory variable having a high importance in the trained model is selected as a variation value, and a graph showing the relation between the selected explanatory variable and the objective variable is generated. This provides an inference result of the variation of the objective variable to the variation of the explanatory variable, which has a large impact on the objective variable, regardless of the user's experience and skill level. This can reduce the possibility that a user fails to consider the critical explanatory variable.

(Item 8) In the data processing apparatus as recited in the above-described Item 7, the variation range is set based on a value of the first explanatory variable included in the training data.

According to the data processing apparatus described in the above-described Item 8, it is possible to set the variation range in which the accuracy of the inference result is guaranteed.

(Item 9) In the data processing apparatus as recited in the above-described Item 7 or 8, a display unit is connected. The display data generation unit generates a plurality of data respectively corresponding to the plurality of first explanatory variables and displays the generated plurality of data on the display unit in descending order of the importance of the corresponding first explanatory variable.

In the data processing apparatus described in the above-described Item 9, in the display unit, graphs are displayed in descending order of the degree of influence of the explanatory variable on the objective variable, and therefore, the graph about the explanatory variable having a large influence on the objective variable can be effectively displayed. Therefore, the usefulness of the inference result can be improved.

(Item 10) In the data processing apparatus as recited in the above-described Item 1 or 2, the inference unit selects at least one of the first explanatory variables from the plurality of explanatory variables, based on an absolute value of a principal component load of each explanatory variable for a particular principal component determined by a principal component analysis of the plurality of explanatory variables. The inference unit predicts the objective variable when and the first explanatory variable is continuously varied within the variation range using the trained model, for each of the selected at least one of the first explanatory variables.

According to the data processing apparatus described in the above-described Item 10, an explanatory variable having a large weight (principal component load) for a particular principal component out of a plurality of explanatory variables to be given to the trained model is selected as variation values, and a graph showing the relation between the selected explanatory variable and the objective variable is generated. This provides an inference result of a variable value of an objective variable to a variation of the explanatory variable having a high contribution to the principal component, regardless of the user's experience and skill. This can reduce the possibility that a user fails to consider a critical explanatory variable.

(Item 11) In the data processing apparatus as recited in the above-described Item 10, the trained model is a model generated by machine learning using training data in which the plurality of explanatory variables is inputs, and the objective variable is a ground truth output. The variation range is set based on a value of the first explanatory variable included in the training data.

According to the data processing apparatus as recited in the above-described Item 11, it is possible to set a variation range in which the accuracy of the inference result is guaranteed.

(Item 12) In the data processing apparatus as recited in the above-described Item 10 or 11, a display unit is connected to the data processing apparatus. The display data generation unit generates a plurality of data respectively corresponding to the plurality of first explanatory variables. The display data generation unit displays the generated plurality of data on the display unit in descending order of the principal component load of the corresponding first explanatory variable.

According to the data processing apparatus as recited in the above-described Item 12, in the display unit, graphs are displayed in descending order of the principal component load of the explanatory variable. Therefore, it is possible to effectively display the graphs on explanatory variables having a high contribution on the principal component. Therefore, the usefulness of the inference result can be improved.

(Item 13) In the data processing apparatus as recited in the above-described Item 1 or 2, a display unit is connected to the data processing apparatus. The inference unit selects each of the plurality of explanatory variables as the first explanatory variable in order, and predicts, by using the trained model, the objective variable when the first explanatory variable is continuously varied within the variation range, for each selected explanatory variable. The display data generation unit generates a plurality of data respectively corresponding to the plurality of explanatory variables. The display data generation unit displays the generated plurality of data on the display unit in descending order of the variation amount of the objective variable.

According to the data processing apparatus as recited in the above-described Item 13, out of the plurality of explanatory variables to be given to the trained model, one having a large variation amount of the objective variable with respect to the variation of the explanatory variable is preferentially selected, and a graph showing the relation between the selected explanatory variable and the objective variable is generated. According to this, regardless of the user's experience and skill level, the explanatory variable having a large influence on the objective variable is automatically selected, and the inference result of the variation of the objective variable with respect to the variation of the explanatory variable is displayed. This can reduce the possibility that a user fails to consider a critical explanatory variable. In addition, the display unit displays the graphs in descending order of the variation amount of the objective variable with respect to the variation of the explanatory variable, and therefore, it is possible to effectively display graphs of the explanatory variable having a large influence on the objective variable. Therefore, the usefulness of inference results can be improved.

(Item 14) In the data processing apparatus as recited in any one of the above-described Items 1 to 13, the inference unit selects two or more of the first explanatory variables from the plurality of explanatory variables. The inference unit predicts, by using the trained model, the objective variable when the first explanatory variable is continuously varied within the variation range, for each of the selected two or more of the first explanatory variables. The display data generation unit generates two more of the data corresponding to the two or more of the first explanatory variables. The data processing apparatus is further provided with a database for storing a type of the first explanatory variable having a largest influence on the objective variable among the two or more of the first explanatory variables in association with information on a project to which the trained model is applied.

According to the data processing apparatus as recited in the above-described Item 14, when training a training model next time, it is possible to select the explanatory variable and the objective variable depending on the project to which the training model for inputting the training data is applied, while referring to the information stored in the database.

(Item 15) The data processing apparatus as recited in the above-described Item 14 is further provided with: a training data generation unit configured to generate training data in which the plurality of explanatory variables are inputs, and the objective variable is a ground truth output; and a training unit configured to generate the trained model by machine learning using the training data. The training data generation unit is configured to provide information on the project and a type of the first explanatory variable associated with the project to a user.

According to the data processing apparatus as recited in the above-described Item 15, the user can select the explanatory variable and the objective variable depending on the project to which the training model for inputting training data is applied. For example, the user can select the objective variable of the trained model with the same project and the explanatory variable having a large influence on the objective variable. With this, since the explanatory variable having a large influence on the objective variable is generated as training data, the usefulness of the trained model for the project can be enhanced.

(Item 16) The data processing apparatus as recited in any one of the above-described Items 1 to 13 is further provided with: a training data generation unit configured to generate training data in which the plurality of explanatory variables are inputs, and the objective variable is a ground truth; a training unit configured to generate the trained model by machine learning using the training date; and a database configured to store the trained model in association with the training data.

According to the data processing apparatus as recited in the above-described Item 16, it is possible to execute the machine learning of a training model and an inference using the trained model by one apparatus.

(Item 17) In the data processing apparatus as recited in the above-described Item 16, the training data generation unit is configured to generate the plurality of training data so as to include a plurality of features extracted using a plurality of data processing conditions different from each other from one data group. The training unit is configured to generate a plurality of trained models from the plurality of training data. The training unit is configured to store each of the plurality of generated trained models in the database in association with corresponding data processing conditions.

According to the data processing apparatus as recited in the above-described Item 17, a plurality of training data different in the data processing condition is generated from one data group, and a plurality of trained models are generated using the plurality of training data. By performing the inference by giving the explanatory variable common to the plurality of trained model, it is possible to know the relation between the data processing condition and the influence of the explanatory variable on the objective variable.

(Item 18) In the data processing apparatus as recited in the above-described Item 17, the inference unit predicts, by using each of the plurality of trained models, the objective variable when the first explanatory variable is continuously varies within the variable range. The display data generation unit generates a plurality of the data indicating a variation of the objective variable with respect to a variable of the first explanatory variable, corresponding to the plurality of trained models.

According to the data processing apparatus as recited in the above-described Item 18, by comparing the generated plurality of data, the user can select a trained model (i.e., suitable data processing condition) considered to be appropriate for considering the relation between the first explanatory variable and the objective variable.

(Item 19) In the data processing apparatus as recited in the above-described Item 18, in a case where one of the data is selected by a user from the plurality of data, the display data generation unit stores the trained model corresponding to the selected data in the database as an appropriate trained model. In a case where a feature is extracted from a data group similar to the one data group, the training data generation unit provides the data processing condition associated with the appropriate trained model to a user.

According to the data processing apparatus as recited in the above-described Item 19, in the training phase, by using the data processing condition determined to be appropriate for considering the relation between the first explanatory variable and the objective variable, it is possible to generate a sample list in which the feature is extracted from the analysis data of the sample. Since the trained model is generated using the training data generated based on the sample list, in the inference phase, the relation between the first explanatory variable to be given to the trained model and the objective variable predicted from the trained model becomes a relation determined to be appropriate by the user. Therefore, it is possible to enhance the usefulness of the inference result.

(Item 20) A inference method according to one aspect of the present invention is configured to predict an object variable from a plurality of explanatory variables by using a trained model. The inference method includes: a step of predicting, by using the trained model, the objective variable when the first explanatory variable is continuously varied within a predetermined variation range in a state in which a first explanatory variable selected from the plurality of explanatory variables is set as a variation value, and second explanatory variables other than the first explanatory variable are set as fixed values; a step of generating data indicating a variation of the objective variable with respect to a variation of the first explanatory variable; and a step of displaying the data generated by the step of generating the data.

According to the inference method as recited in the above-described Item 20, the user can easily visually predict how the objective variable varies when the first explanatory variable serving as a variation value is continuously varied, based on the displayed data. Therefore, the usefulness of the inference result can be enhanced.

Although some embodiments of the present invention have been described, the embodiments disclosed herein are to be considered in all respects as illustrative and not restrictive. The scope of the present invention is indicated by the above-described Items, and it is intended to include all modifications within the meanings and ranges equivalent to those of the claims.

1. A data processing apparatus comprising:
an inference unit configured to predict an objective variable from a plurality of explanatory variables by using a trained model; and
a display data generation unit configured to generate data for displaying an inference result by the inference unit,
wherein the inference unit is configured to
set a first explanatory variable selected from the plurality of explanatory variables as a variation value and set second explanatory variables other than the first explanatory variable as fixed values, and
predict, by using the trained model, the objective variable when the first explanatory variable is continuously varied within a predetermined variation range, and
wherein the display data generation unit generates data indicating a variation of the objective variable with respect to a variation of the first explanatory variable.

2. The data processing apparatus as recited in claim 1,
wherein the display data generation unit generates a two-dimensional graph in which the first explanatory variable is represented by a first axis, and the objective variable is represented by a second axis.

3. The data processing apparatus as recited in claim 2,
wherein the inference unit is configured to
select two or more of the first explanatory variables from the plurality of explanatory variables, and
predict, by using the trained model, the objective variable when the first explanatory variable is continuously varied within the variation range, for each of the selected two or more of the first explanatory variables,
wherein a display unit is connected to the data processing apparatus, and
wherein the display data generation unit is configured to
generate two or more of the two-dimensional graphs corresponding to the two or more of the first explanatory variables, and
display the generated two or more of the two-dimensional graphs on the display unit in a superimposed manner.

4. The data processing apparatus as recited in claim 1,
wherein the display data generation unit is configured to provide a first user interface for selecting the first explanatory variable and setting the variation range, and
wherein the first user interface includes information on a recommendation range of the variation range.

5. The data processing apparatus as recited in claim 4,
wherein the trained model is a model generated by machine learning using training data in which the plurality of explanatory variables are inputs, and the objective variable is a ground truth output, and
wherein the recommendation range is set based on a value of the first explanatory variable included in the training data.

6. The data processing apparatus as recited in claim 4 or 5,
wherein the display data generation unit is configured to further provide a second user interface for setting a value of the second explanatory variable.

7. The data processing apparatus as recited in claim 1,
wherein the trained model is a model generated by machine learning using training data in which the plurality of explanatory variables are inputs, and the objective variable is a ground truth output, and
wherein the inference unit is configured to
select at least one of the first explanatory variables from the plurality of explanatory variables based on importance of each explanatory variable in the trained model, and
predict, by using the trained model, the objective variable when the first explanatory variable is continuously varied within the variation range, for each of the selected at least one of the first explanatory variables.

8. The data processing apparatus as recited in claim 7,
wherein the variation range is set based on a value of the first explanatory variable included in the training data.

9. The data processing apparatus as recited in claim 7 or 8,
wherein a display unit is connected to the data processing apparatus, and
wherein the display data generation unit is configured to
generate a plurality of the data respectively corresponding to the plurality of first explanatory variables, and
display the generated plurality of data on the display unit in descending order of the importance of the corresponding first explanatory variables.

10. The data processing apparatus as recited in claim 1,
wherein the inference unit is configured to
select at least one of the first explanatory variables from the plurality of explanatory variables, based on an absolute value of a principal component load of each explanatory variable with respect to a particular principal component determined by a principal component analysis of the plurality of explanatory variables, and
predict, by using the trained model, the objective variable when the first explanatory variable is continuously varied within the variation range, for each of the selected at least one of the first explanatory variables.

11. The data processing apparatus as recited in claim 10,
wherein the trained model is a model generated by machine learning using training data in which the plurality of explanatory variables is inputs, and the objective variable is a ground truth output, and
wherein the variation range is set based on a value of the first explanatory variable included in the training data.

12. The data processing apparatus as recited in claim 10,
wherein a display unit is connected to the data processing apparatus, and
wherein the display data generation unit is configured to
generate a plurality of data corresponding to the plurality of first explanatory variables, and
display the generated plurality of data on the display unit in descending order of the principal component load of the corresponding first explanatory variable.

13. The data processing apparatus as recited in claim 1 or 2,
wherein a display unit is connected to the data processing apparatus,
wherein the inference unit is configured to select each of the plurality of explanatory variables as the first explanatory variable in order, and
predict, by using the trained model, the objective variable when the first explanatory variable is continuously varied within the variation range, for each selected explanatory variable, and
wherein the display data generation unit is configured to
generate a plurality of the data corresponding to the plurality of explanatory variables, and
display the generated plurality of data on the display unit in descending order of the variation amount of the objective variable.

14. The data processing apparatus as recited in claim 1 or 2,
wherein the inference unit is configured to
select two or more of the first explanatory variables from the plurality of explanatory variables, and
predict, by using the trained model, the objective variable when the first explanatory variable is continuously varied within the variation range, for each of the selected two or more of the first explanatory variables, and
wherein the display data generation unit generates two more of the data corresponding to the two or more of the first explanatory variables, and
wherein the data processing apparatus further comprises a database for storing a type of the first explanatory variable having a largest influence on the objective variable among the two or more of the first explanatory variables in association with information on a project to which the trained model is applied.

15. The data processing apparatus as recited in claim 14, further comprising:
a training data generation unit configured to generate training data in which the plurality of explanatory variables are inputs, and the objective variable is a ground truth output; and
a training unit configured to generate the trained model by machine learning using the training data,
wherein the training data generation unit is configured to provide information on the project and a type of the first explanatory variable associated with the project to a user.

16. The data processing apparatus as recited in claim 1, further comprising:
a training data generation unit configured to generate training data in which the plurality of explanatory variables are inputs, and the objective variable is a ground truth;
a training unit configured to generate the trained model by machine learning using the training date; and
a database configured to store the trained model in association with the training data.

17. The data processing apparatus as recited in claim 16,
wherein the training data generation unit is configured to generate the plurality of training data so as to include a plurality of features extracted using a plurality of data processing conditions different from each other from one data group,
wherein the training unit is configured to
generate a plurality of trained models from the plurality of training data; and
store each of the plurality of generated trained models in the database in association with corresponding data processing conditions.

18. The data processing apparatus as recited in claim 17,
wherein the inference unit predicts, by using each of the plurality of trained models, the objective variable when the first explanatory variable is continuously varies within the variable range, and
wherein the display data generation unit generates a plurality of the data indicating a variation of the objective variable with respect to a variable of the first explanatory variable, corresponding to the plurality of trained models.

19. The data processing apparatus as recited in claim 18,
wherein in a case where one of the data is selected by a user from the plurality of data, the display data generation unit stores the trained model corresponding to the selected data in the database as an appropriate trained model, and
wherein in a case where a feature is extracted from a data group similar to the one data group, the training data generation unit provides the data processing condition associated with the appropriate trained model to a user.

20. An inference method of predicting an objective variable from a plurality of explanatory variables by using a trained model, comprising the steps of:
predicting, by using the trained model, the objective variable when the first explanatory variable is continuously varied within a predetermined variation range in a state in which a first explanatory variable selected from the plurality of explanatory variables is set as a variation value, and second explanatory variables other than the first explanatory variable are set as fixed values;
generating data indicating a variation of the objective variable with respect to a variation of the first explanatory variable; and
displaying the data generated by the step of generating the data.
